(19) United States
(12) Patent Application Publication
(10) Pub. No.: US 2021/0000955 A1
(43) Pub. Date: Jan. 7, 2021

(54) PHOTODYNAMICALLY ACTIVE ORGANOSILICA NANOPARTICLES AND MEDICAL USES THEREOF

(71) Applicant: NANYANG TECHNOLOGICAL UNIVERSITY, SIngapore (SG)

(72) Inventors: Huijun Phoebe THAM, Singapore (SG); Keming XU, Singapore (SG); Wei Qi LIM, Singapore (SG); Hongzhong CHEN, Singapore (SG); Subramanian VENKATRAMAN, Singapore (SG); Chenjie XU, Singapore (SG); Yanli ZHAO, Singapore (SG)

(21) Appl. No.: 16/770,543

(22) PCT Filed: Dec. 5, 2018
(86) PCT No.: PCT/SG2018/050593

§ 371 Date: Jun. 5, 2020

(30) Foreign Application Priority Data
Dec. 8, 2017 (SG) 10201710241V

(51) Int. Cl. A61K 41/00; A61K 47/24; A61K 47/69; A61K 31/506; A61K 31/519

(52) U.S. Cl. CPC A61K 41/0071; A61K 47/24; A61K 47/6929; A61K 9/0009; A61K 31/506; A61K 31/519; A61K 47/6955

(57) ABSTRACT

The present application provides an organosilica nanoparticle comprising: (a) a photosensitizer for photodynamic therapy covalently incorporated therein; and (b) optionally, at least one agent encapsulated therein, as well as a pharmaceutical composition comprising said organosilica nanoparticle. Also provided herein are said organosilica nanoparticle or pharmaceutical composition for use as a medicament or in the treatment of a disease, disorder, or condition. More specifically, provided is a method for treating a disease, disorder, or condition in a subject using said aid organosilica nanoparticle or pharmaceutical composition.

### CROSS-REFERENCE TO RELATED APPLICATION

This application makes reference to and claims the benefit of priority of the Singapore Patent Application No. 10201710241V filed on 08 Dec. 2017, the content of which is incorporated herein by reference for all purposes, including an incorporation of any element or part of the description, claims or drawings not contained herein and referred to in Rule 20.5(a) of the PCT, pursuant to Rule 4.18 of the PCT.

### FIELD OF THE INVENTION

The present invention relates generally to photodynamically active organosilica nanoparticles comprising a photosensitizer and encapsulating at least one agent, pharmaceutical compositions comprising said organosilica nanoparticles, as well as medical applications using said organosilica nanoparticles or pharmaceutical compositions.

### BACKGROUND OF THE INVENTION

Photodynamic therapy (PDT) has been widely utilized to treat malignant tumors. PDT involves the utilization of photosensitizers that generate singlet oxygen species upon light irradiation to eliminate malignant cells near the irradiated site, while sparing systemic toxicity. The advantages of PDT include noninvasiveness, high selectivity, and minimized side effects or damage to cells away from the irradiated site. To achieve a high therapeutic efficacy in PDT, it is crucial to deliver highly potent photosensitizers to the disease site. In clinical practice, photosensitizers have been administered systemically through intravenous injections. However, such a drug administration approach would result in whole-body distribution of photosensitizers while needing a much higher amount to be administered. Photosensitizers would also require modification in order to circulate longer in the bloodstream.

Transdermal drug delivery is an attractive means for drug administration, given its non/minimally-invasive nature, high patient compliance, and direct route of entry bypassing gastrointestinal or liver metabolism. It is especially attractive for skin-related malignancies (i.e.

skin cancer). Therefore, topical delivery of photosensitizers for treating superficial basal cell carcinomas and actinic keratosis through PDT is desirable.

Some recent studies reported formulating photosensitizers using nanoparticles (e.g., gold nanoparticles and micelles) to enhance the PDT efficiency, increase the circulation time through systemic delivery, and co-deliver two and more drugs. For example, liposomes were reported to encapsulate 5-aminolevulinic or temoporfin in the topical treatment of skin cancers.

Unfortunately, limitations such as low biocompatibility and stability of nanoparticles, low drug loading capacity, and lack of effective drug-photosensitizer combination still exist. More importantly, all these formulations are short of transdermal penetration ability, because hydrophilic systems and macromolecules (>500 Da) cannot diffuse through compact lipid-rich matrix of the stratum corneum.

Therefore, there is still need in the art for improved technologies that overcome the drawbacks of existing techniques.

### SUMMARY OF THE INVENTION

The present invention satisfies the aforementioned need in the art by providing novel organosilica nanoparticles and pharmaceutical compositions as well as methods of using the same.

In a first apect, the invention provides an organosilica nanoparticle comprising: (a) a photosensitizer for photodynamic therapy covalently attached thereto; and (b) optionally, at least one agent encapsulated therein.

In various embodiments, the organosilica nanoparticle is a mesoporous organosilica nanoparticle and the photosensitizer is incorporated within the framework of the nanoparticle.

In various embodiments, the organosilica nanoparticle is less than 50 nm in diameter.

In various embodiments, the organosilica nanoparticle is formed by condensation of the photosensitizer with an alkoxysilane, preferably a di- tri- or tetraalkoxysilane, more preferably tetramethoxysilane (TMOS) or tetraethoxysilane (TEOS).

In various embodiments, the photosensitizer is modified with a silicon-containing group of the formula —Si(OR₆)ₓ(R₇)₃₋ₓ, wherein R₆ and R₇ are independently selected from C₁-C₄ alkyl and C₂-C₄ alkenyl groups, preferably methyl or ethyl, and x is 0, 1, 2, or 3, preferably 2 or 3.

In various embodiments, the photosensitizer is a reaction product of phthalocyanine with an alkoxysilane of the formula A—(CH₂)_{y}—Si(OR₆)ₓ(R₇)₃₋ₓ, wherein A is a group reactive with phthalocyanine, preferably selected from —NCO, —COOH, —OH, and epoxy, x is 0, 1, 2, or 3, preferably 2 or 3, and y is 1, 2, or 3, preferably 3.

In various embodiments, the photosensitizer is a phthalocyanine compound of formula (I) or (I′),

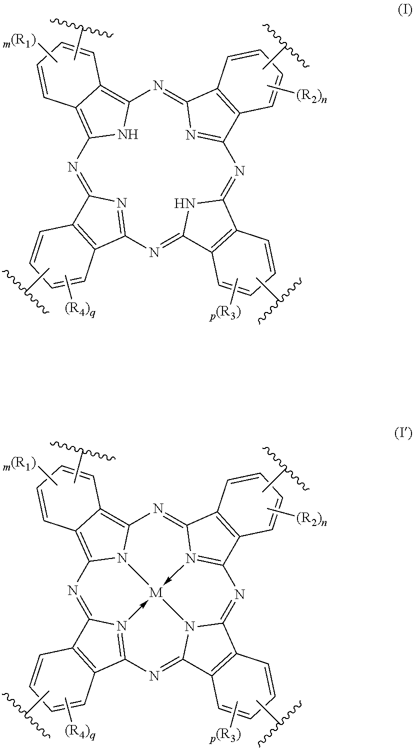

wherein:
M is a metal selected from the group consisting of Zn, Al, Cu, Fe, Mn, Mo, Ni, and V, preferably Zn;
R₁, R_{2,} R_{3,} and R₄ are each independently C₁-C₆ alkyl;
m, n, p, and q are each independently 0, 1, 2, or 3; and [symbol] represents a group of formula —NH—B—, wherein B is a silicon-containing linker group that is covalently integrated into the framework of the nanoparticle.

In various embodiments, the organosilica nanoparticle is obtainable using (a) an organosilica precursor of formula (II) or (II'); and (b) an inorganic silica source selected from the group consisting of TMOS, TEOS, and combinations thereof, via silane co-condensation and hydrolysis,

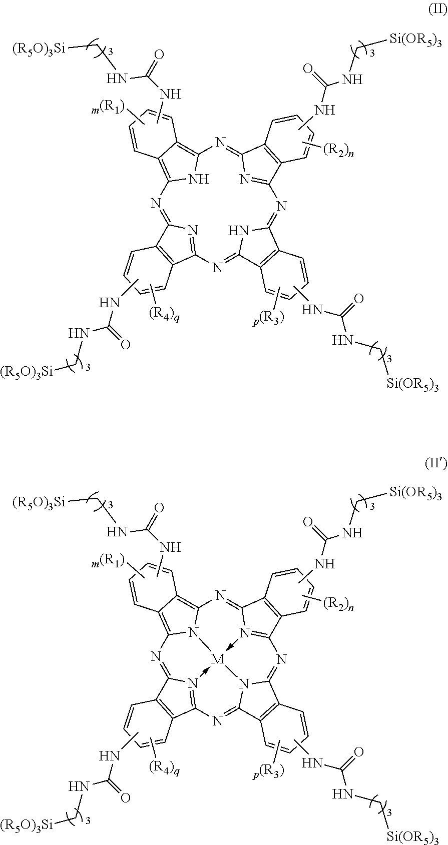

wherein:
M is a metal selected from the group consisting of Zn, Al, Cu, Fe, Mn, Mo, Ni, and V, preferably Zn;
R₁, R_{2,} R_{3,} R_{4,} and R5 are each independently C₁-C₆ alkyl; and
m, n, p, and q are each independently 0, 1, 2, or 3.

In various embodiments, m, n, p, and q are 0.

In various embodiments, R₅ is CH₂CH_{3.}

In various embodiments, R₅ is CH₂CH_{3,} and m, n, p, and q are 0.

In various embodiments, the inorganic silica source is TMOS.

In various embodiments, the molar ratio of the organosilica precursor of formula (II) or (II′) and the inorganic silica source used for the synthesis of the nanaoparticle is between 1:100 and 1:1000, preferably between 1:200 and 1:500, more preferably between 1:250 and 1:300, most preferably 1:270.

In various embodiments, the at least one agent is a compound for the treatment or prevention of a disease, disorder, or condition.

In various embodiments, the disease, disorder, or condition is selected from the group consisting of primary melanoma, metastasized melanoma, basal cell carcinoma, Bowen's disease, actinic keratosis, abnormal scarring (keloid and hypertrophic scars), atoptic dermatitis, warts, pre-malignant non-melanoma skin lesions, and cholangiocarcinoma.

In various embodiments, the at least one agent is selected from the group consisting of antibiotics, steroids, chemotherapeutic drugs, immunomodulators, anti-inflammatory agents, drugs for the treatment of cancer such as BRAF inhibitors, therapeutic peptides or proteins or monoclonal antibodies such as anti-CTLA4 or anti-PD-1 antibodies, siRNAs, and plasmids, or combinations thereof.

In various embodiments, the at least one agent is selected from the group consisting of dabrafenib, trametinib, camptothecin, doxorubicin, and combinations thereof.

In various embodiments, the disease, disorder, or condition is melanoma and the at least one agent is dabrafenib and/or trametinib.

In a second aspect, the invention provides a pharmaceutical composition comprising an organosilica nanoparticle disclosed herein and a pharmaceutically acceptable carrier.

In various embodiments, the pharmaceutical composition is a topical formulation.

In a third aspect, the invention provides an organosilica nanoparticle or pharmaceutical composition disclosed herein for use as a medicament.

In a fourth aspect, the invention provides an organosilica nanoparticle or pharmaceutical composition disclosed herein for use in the treatment of a disease, disorder, or condition, preferably cancer, more preferably skin cancer, most preferably melanoma.

In a fifth aspect, the invention provides a method for treating a disease, disorder, or condition in a subject, comprising the steps of:

(a) administering, preferably topically, to the subject a therapeutically effective amount of an organosilica nanoparticle or pharmaceutical composition disclosed herein; and (b) exposing the subject to photoirradiation, thereby treating the disease, disorder, or condition at least partly by photodynamic therapy.

In various embodiments, the photoirradiation is by near-infrared light, preferably by 730-nm laser.

In various embodiments, the disease, disorder, or condition is selected from the group consisting of primary melanoma, metastasized melanoma, basal cell carcinoma, Bowen's disease, actinic keratosis, abnormal scarring (keloid and hypertrophic scars), atoptic dermatitis, warts, pre-malignant non-melanoma skin lesions, and cholangiocarcinoma.

In various embodiments, the disease, disorder, or condition is a skin cancer, preferably melanoma, and the organosilica nanoparticle or pharmaceutical composition is administered topically.

In various embodiments, the method comprises enhancing skin penetration of the organosilica nanoparticle using a microneedle patch.

In various embodiments, the subject is a human or mammal.

### BRIEF DESCRIPTION OF THE DRAWINGS

The invention will be better understood with reference to the detailed description when considered in conjunction with the non-limiting examples and the accompanying drawings.

FIG. 1. a) Scheme for synthesis of PcNP@Drug and its penetration into diseased skin. b) Cellular mechanism for the action of PcNP@Drug.

FIG. 2. Characterization of PcNP. a) TEM image of PcNP, scale bar=50 nm. Inset: magnified image of an individual PcNP, scale bar=20 nm. b) Absorbance curve of Pc-Si and PcNP. c)

DLS measurement of purified PcNP and final PcNP@Drug. d) XPS spectra of PcNP, indicating O, N, C and Si peaks from PcNP. e) High resolution scan of nitrogen binding energy for PcNP. f) Cumulative drug release kinetics of dabrafenib and trametinib from PcNP@Drug at pH 7.4 and 5, *p<0.05.

FIG. 3. Characterization of PcNP. Absorbance curves of a) PcNP and b) Pc-Si upon laser irradiation, reflecting the photostability of PcNP. Inset of a): Absorbance curves of PcNP between 600-900 nm. c) Percentage absorbance at 722 nm plotted against irradiation time for PcNP and Pc-Si. d) N₂ adsorption and desorption patterns of PcNP and PcNP@Drug measured from P/Pₒ=0.05 to 0.2. e) Pore size distributions of PcNP and PcNP@Drug measured by DFT analysis. f) Photothermal behavior of PcNP at 2, 0.5, 0.1 and 0 mg/mL concentrations upon 730 nm, 1 W/cm² laser irradiation.

FIG. 4. In vitro experiments of PcNP and PcNP@Drug. a) Time-dependent cellular internalization of PcNP in A375 cells after incubation for 0.5 h, 2 h, and 4 h. Blue channel: Hoechst 33342 filter indicating nucleus location. Red channel: nanovehicle location. Xex: 488 and 561 nm, Xem: 565-700 nm. Scale bar=20 pm. b) Live/dead cellular imaging of SKMEL-28 cell line for PcNP+hv (PDT), PcNP@Drug-hv (targeted therapy), and PcNP@Drug+hv (combinational treatment). Cells were incubated with PcNP or PcNP@Drug for 4 hours. Irradiation: 730 nm, 0.5 W/cm² laser for 15 minutes. λₑₓ: 640 nm, λₑₘ: 650-700 nm. Scale bar=200 μm. Corresponding cell viability of c) A375, d) SKMEL-28, e) HDF, and f) B16F10 cell lines. Incubation time: 16 hours, irradiation of 8 min/well.

FIG. 5. Mechanisms of cell deaths. a) In vitro cellular oxidative stress imaging of SKMEL-28 cell line for PcNP in the absence (PcNP-hv) and presence (PcNP+hv) of NIR light. Hoechst 33342 channel indicates nucleus location, and carboxy-H2DCFDA channel indicates the presence of oxidative stress. Scale bar=200 μm. b) Detection of caspase 3 activity in A375 cells upon various nanovehicle treatments. *p<0.005.

FIG. 6. Efficacy of PcNP on 3D tumor spheroids. a) Microscopic images of representative tumor spheroids receiving different treatments upon time. Scale bar=500 μm. b) Relative tumor size chart. Error bar represents standard error of mean, *p<0.05, n=5. c) Viability of tumor spheroids conducted using acid phosphatase assay. PcNP+hv (group receiving PDT treatment), PcNP@Drug-hv (group receiving targeted therapy treatment), and PcNP@Drug+hv (group receiving combination PDT and targeted therapy treatment). Error bar represents standard deviation, *p<0.05, **p<0.001, n=5.

FIG. 7. Topical penetration of PcNP on porcine skin. a) Fluorescence of penetrated PcNP (20 mg/mL) versus free Pc on fresh porcine skin for 10-minute and 1-hour durations with and without the microneedle (MN) assistance. Luminescence was recorded on an IVIS machine. λₑₓ: 620 nm, λₑₘ: 640-700 nm. b) Intensity of the luminescence on the porcine skin tabulated into a graph. **p<0.01. c) Cross-section images of porcine skin penetrated with PcNP (20 mg/mL) for (i) 10 minutes without MN, ii) 10 minutes with MN, iii) 1 hour without MN, and iv) 1 hour with MN. Scale bar=200 μm. The skin was obtained from the rib portion of a pig, which was sectioned and fixed prior to the imaging. d) Fluorescence intensity of skin sections penetrated with PcNP (20 mg/mL) for 10 min and 1 hour, with and without MN. *p<0.05, **p<0.01, ***p<0.005, n=4.

FIG. 8. In vivo antitumoral efficacy of PcNP. a) Relative tumor size growth chart for control, PcNP+hv (group receiving PDT treatment), PcNP@Drug-hv (group receiving targeted therapy treatment), and PcNP@Drug+hv (group receiving combination treatment). The tumor sizes were measured together with the mouse skin using a digital caliper. Green arrow: nanovehicle treatment, red arrow: laser treatment. *p<0.05, n=5. b) Body weights of mice throughout the duration of treatments. c) Photographs of excised tumors on day 16 showing relative sizes of each group. d) Weights of tumors excised from the mice in different groups. The tumors were extracted out, followed by the measurement. *p<0.05, **p<0.01. e) H&E stained images of tumor cross-sections indicating cell nuclei density.

FIG. 9. Reaction scheme of Pc-4NH₂ with 3-(triethoxysilyl)propyl isocyanate to form Pc-Si.

FIG. 10. Optimization for the ratio of TMOS to Pc. a) Actual amount of Pc loaded in nanoparticles (right axis, dotted line) by corresponding to the theoretical amount (solid line) as determined by elemental analysis. b) Singlet oxygen production efficiency tested using ABDA.

FIG. 11. Singlet oxygen quantum yield calculation. Quenching of DPBF when a) methylene blue and d) PcNP were mixed with DPBF of similar optical density. Irradiation conditions were 730 nm, 1 W/cm² laser. First order exponential fitting of DPBF absorbance at 423 nm for b) methylene blue and e) PcNP. c) Absorbance curves for PcNP and methylene blue. f) Excitation-dependent luminescence of PcNP displaying peak fluorescence at excitation of 638 nm. g) TEM image of PcNP@Drug. Scale bar=50 nm.

FIG. 12. Zeta potential of a) PcNP and b) PcNP@Drug.

FIG. 13. Drug loading capacity (DLC) and encapsulation efficiency (EE) of a) dabrafenib, b) trametinib and c) dabrafenib +trametinib combination, into PcNP.

FIG. 14. Cytotoxicity of PcNP at various concentrations over 48 hours, tested on a) A375, b) B16-F10, c) SKMEL-28, d) HDF, and e) HEK cell lines.

FIG. 15. In vitro dosage optimization results. Various ratios of dabrafenib to trametinib (1:0, 150:1, 50:1, 1:1) tested on a) SKMEL-28, b) B16-F10, c) A375, d) HDF, and e) HEK cell lines.

FIG. 16. Time-dependent internalization of PcNP@Drug in A375 cells. Scale bar=20 μm.

FIG. 17. Cell viability studies of free drug+free Pc solution on a) 2D A375 cells using MTT and b) 3D A375 spheroids using acid phosphatase assay.

FIG. 18. Quantitative measurement for the number of oxidatively stressed cells vs number of cells present.

FIG. 19. Photographs of mice in different experimental groups over the treatment period.

FIG. 20. Relative tumor growth curves of mice treated with free Pc+free drug mixture without MN, and the untreated mice as control.

FIG. 21. Tumor growth inhibition (TGI) values of PcNP+hv, PcNP@Drug-hv, and PcNP@Drug+hv treatment groups.

FIG. 22. Immunohistochemical characterization (TUNEL staining) of tumor tissues after mice were treated with PBS (control), PcNP+hv, PcNP@Drug-hv, or PcNP@Drug+hv. a) Representative TUNEL staining images, scale bar=100 μm. b) Quantitative results of TUNEL positive cells in tumor tissues (n=5). **, p<0.01; ***, p<0.001.

### DETAILED DESCRIPTION OF THE INVENTION

The following detailed description refers to, by way of illustration, specific details and embodiments in which the invention may be practiced. These embodiments are described in sufficient detail to enable those skilled in the art to practice the invention. Other embodiments may be utilized and structural, and logical changes may be made without departing from the scope of the invention. The various embodiments are not necessarily mutually exclusive, as some embodiments can be combined with one or more other embodiments to form new embodiments.

Unless otherwise defined, all technical and scientific terms used herein have the same meaning as commonly understood by one of ordinary skill in the art. The singular terms “a,” “an,” and “the” include plural referents unless context clearly indicates otherwise. Similarly, the word “or” is intended to include “and” unless the context clearly indicates otherwise. The term “comprises” means “includes.” In case of conflict, the present specification, including explanations of terms, will control.

The inventors have surprisingly found that an organosilica nanoparticle comprising a photosensitizer such as phthalocyanine covalently attached thereto is suitable for photodynamic therapy of a disease, disorder, or condition such as melanoma, and that agents encapsulated in the organosilica nanoparticle can act in synergism with the photodynamic therapy. Therefore, such organosilica nanoparticles can be used as a novel medicament.

In a first apect, the invention provides an organosilica nanoparticle comprising: (a) a photosensitizer for photodynamic and/or photothermal therapy covalently attached thereto;

and

(b) optionally, at least one agent encapsulated therein.

The term “nanoparticle” as used herein refers to any particle having a size from 10 to 250 nm. The diameter of the nanoparticle as described herein can range in the size from 10 nm to 250 nm; 10 nm to 200 nm; 10 nm to 160 nm; 10 nm to 140 nm; 10 nm to 120 nm; 10 nm to 100 nm; 10 nm to 80 nm; 10 nm to 60 nm; 10 nm to 50 nm; 20 nm to 250 nm; 30 nm to 250 nm; 40 nm to 250 nm; 80 nm to 250 nm; 100 nm to 250 nm; or 150 nm to 250 nm. In preferred embodiments, the nanoparticles are less than 50 nm in diameter. A nanoparticle may have a variety of shapes and cross-sectional geometries.

The term “organosilica” as used herein refers to an organosiloxane compound that comprises one or more organic groups bound to two or more Si atoms. It is used herein in relation to the nanoparticles to refer to particles comprising an organosiloxane compound.

The term “photosensitizer” as used herein refers to molecules, which upon irradiation with light having a wavelength corresponding at least in part to the absorption bands of said “photosensitizer” interact through energy transfer with another molecule to produce radicals, and/or singlet oxygen, and/or ROS. For example, in its excited state, the photosensitizer can undergo intersystem crossing and transfer energy to oxygen in tissues being treated by photodynamic therapy to produce ROS, such as singlet oxygen. Photosensitizing molecules are well-known in the art and include lead compounds, including but not limited to, chlorines, chlorophylls, coumarines, cyanines, fullerenes, metallophthalocyanines, metalloporphyrins, methylenporphyrins, naphthalimides, naphthalocyanines, nile blue, perylenequinones, phenols, pheophorbides, pheophyrins, phthalocyanines, porphycenes, porphyrins, psoralens, purpurins, quinines, retinols, rhodamines, thiophenes, verdins, xanthenes, and dimers and oligomers thereof. The term “photosensitizer” also includes photosensitizer derivatives; for example, the positions in a photosensitizer may be functionalized by an alkyl, functional group, peptide, protein, or nucleic acid or a combination thereof.

The term “photodynamic therapy” refers to a process whereby light of a specific wavelength is directed to tissues or cells undergoing treatment that have been rendered photosensitive through the administration of a photosensitizer. This term should be interpreted broadly to encompass “photothermal therapy”, i.e. treatment by generating heat upon exposure of the photosensitizer to light.

In the present application, the photosensitizer is covalently attached to the organosilica nanoparticle. The term “covalently attached” is used interchangeable with “covalently bonded” and refers to the forming of a chemical bonding that is characterized by the sharing of pairs of electrons between atoms. As photodynamic therapy does not require the release of photosensitizers, the covalent linkage of photosensitizers in the silica matrix can allow sufficient loading of photosensitizers and yet prevent their aggregation-induced quenching, thus increasing the quantum yield of photosensitizers in the system.

The term “agent” as used herein refers to a chemical compound, a biological macromolecule (such as a nucleic acid, an antibody, an antibody fragment, a protein, or a peptide), or a combination thereof. The activity of such agents may render them suitable as a therapeutic agent which is a biologically, physiologically, or pharmacologically active substance that acts locally or systemically in a subject. An organosilica nanoparticle disclosed herein may comprise one or more such agents.

In preferred embodiments, the organosilica nanoparticle of the invention is a mesoporous organosilica nanoparticle and the photosensitizer is incorporated within the framework of the nanoparticle, i.e. the photosensitizer is covalently attached to the inorganic framework of the mesoporous organosilica nanoparticle and forms an integral part thereof.

The term “mesoporous organosilica nanoparticle” is also known in the art as periodic mesoporous organosilicas (PMOs), a class of organic-inorganic polymers characterized by highly ordered pores presenting a large surface area. These materials also exhibit low cytotoxicity, tuneable pore size, and are biodegradable. The pores may have a diameter of between 0.05 nm to 10 nm, preferably between 1 nm to 8 nm, more preferably 2.5 nm to 5 nm.

In various embodiments, the organosilica nanoparticle is formed by condensation of the photosensitizer with with an alkoxysilane, preferably a di- tri- or tetraalkoxysilane, more preferably TMOS or TEOS.

In various embodiments, the photosensitizer is modified with a silicon-containing group of the formula —Si(OR₆)x(R₇)₃₋ₓ, wherein R₆ and R₇ are independently selected from C₁-C₄ alkyl and C₂-C₄ alkenyl groups, preferably methyl or ethyl, and x is 0, 1, 2, or 3, preferably 2 or 3.

In various embodiments, the photosensitizer is a reaction product of phthalocyanine with an alkoxysilane of the formula A—(CH₂)_{y}—Si(OR₆)ₓ(R₇)₃₋ₓ, wherein A is a group reactive with phthalocyanine, preferably selected from —NCO, —COOH, —OH, and epoxy, x is 0, 1, 2, or 3, preferably 2 or 3, and y is 1, 2, or 3, preferably 3.

The term “phthalocyanine” as used herein refers to any compound belonging to the general class of macrocyclic phthalocyanines, and includes naphthalocyanine, quinolinephthalocyanines etc, as well as substituted derivatives thereof. Such phthalocyanines include metal-free phthalocyanines and, further, phthalocyanines containing metals such as Zinc, aluminum, copper, iron, manganese, molybdenum, nickle, and vanadium.

In preferred embodiments, the photosensitizer is a phthalocyanine compound of formula (I) or (I′),

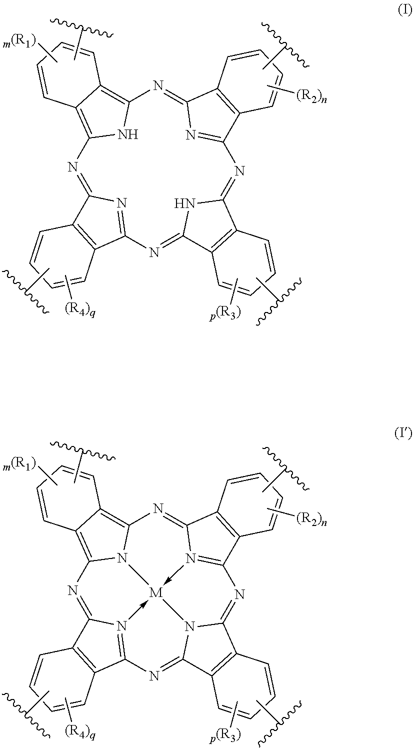

wherein:

M is a metal selected from the group consisting of Zn, Al, Cu, Fe, Mn, Mo, Ni, and V, and Ni, preferably Zn;

R₂, R₃, and R₄ are each independently C₁-C₆ alkyl;

m, n, p, and q are each independently 0, 1, 2, or 3; and

[symbol] represents a group of formula —NH—B—, wherein B is a silicon-containing linker group that is covalently integrated into the framework of the nanoparticle.

B can be any silicon-containing functional group or moiety that forms linkage between the photosensitizer and the framework of the nanoparticle.

The organosilica nanoparticle disclosed herein may be prepared using any methods known in the art.

In preferred embodiments, the organosilica nanoparticle is obtainable using (a) an organosilica precursor of formula (II) or (II′); and (b) an inorganic silica source selected from the group consisting of TMOS, TEOS, and combinations thereof, via silane co-condensation and hydrolysis.

The term “co-condensation and hydrolysis” as used herein refers to a standard sol-gel process of alkoxysilanes. The most frequently used condensable inorganic precursor which builds the network via the formation of siloxane bonds are TMOS/TEOS. Other silica sources may also be used, such as water glass, amorphous silica and kanemite, but the resultant materials may be suboptimal for therapeutic applications. See, for example, Hoffmann & Froba, Chem. Soc. Rev., 2011, 40, 608-620, the disclosure of which is incorporated herein by reference in its entirety.

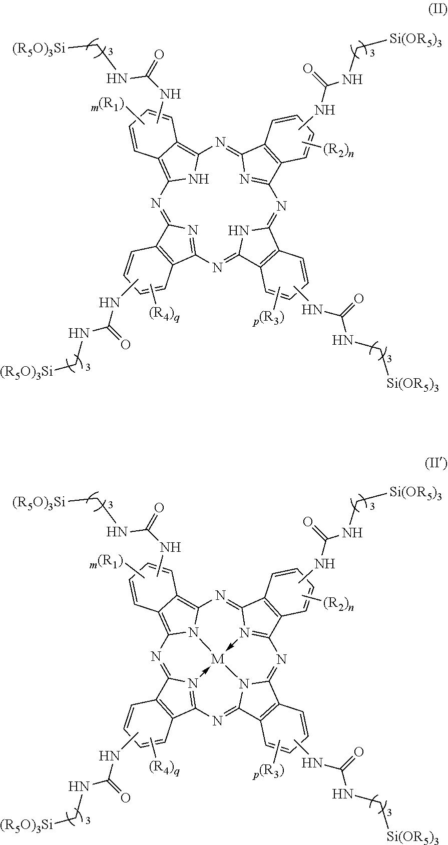

wherein:
M is a metal selected from the group consisting of Zn, Al, Cu, Fe, Mn, Mo, Ni, and V, preferably Zn;

R₁, R₂, R₃, R₄, and R₅ are each independently C₁-C₆ alkyl; and

m, n, p, and q are each independently 0, 1, 2, or 3.

In various embodiments, m, n, p, and q are 0. In various embodiments, R₅ is CH₂CH₃. In preferred embodiments, R₅ is CH₂CH₃, and m, n, p, and q are 0.

In various embodiments, the inorganic silica source is TMOS. In preferred embodiments, R₅ is CH₂CH₃, m, n, p, and q are 0, and the inorganic silica source is TMOS.

In various embodiments, the molar ratio of the organosilica precursor of formula (II) or (II′) (e.g. when R₅ is CH₂CH₃, and m, n, p, and q are 0) and the inorganic silica source (e.g. TMOS) used for the synthesis of the nanaoparticle is between 1:100 and 1:1000, preferably between 1:200 and 1:500, more preferably between 1:250 and 1:300, most preferably 1:270.

In various embodiments, the at least one agent is a compound for the treatment or prevention of a disease, disorder, or condition.

The term “treat” as used herein refers to partially or completely alleviating, ameliorating, improving, relieving, delaying onset of, inhibiting progression of, reducing severity of, and/or reducing incidence of one or more symptoms or features of a particular disease, disorder, or condition. The agent may also be administered to a subject who does not exhibit signs of a disease, disorder, or condition for prevention thereof and/or to a subject who exhibits only early signs of a disease, disorder, or condition for the purpose of decreasing the risk of developing pathology associated with the disease, disorder, or condition.

In various embodiments, the disease, disorder, or condition is selected from the group consisting of primary melanoma, metastasized melanoma, basal cell carcinoma, Bowen's disease, actinic keratosis, abnormal scarring (keloid and hypertrophic scars), atoptic dermatitis, warts, pre-malignant non-melanoma skin lesions, and cholangiocarcinoma.

In various embodiments, the at least one agent is selected from the group consisting of antibiotics, steroids, chemotherapeutic drugs, immunomodulators, anti-inflammatory agents, drugs for the treatment of cancer such as BRAF inhibitors, therapeutic peptides or proteins or monoclonal antibodies such as anti-CTLA4 or anti-PD-1 antibodies, siRNAs, and plasmids, or combinations thereof.

In various embodiments, the at least one agent is selected from the group consisting of dabrafenib, trametinib, camptothecin, doxorubicin, and combinations thereof.

In various embodiments, the disease, disorder, or condition is melanoma and the at least one agent is dabrafenib and/or trametinib.

In a second aspect, the invention provides a pharmaceutical composition comprising an organosilica nanoparticle disclosed herein and a pharmaceutically acceptable carrier.

The term “pharmaceutical composition” refers to a composition that is, within the scope of sound medical judgment, suitable for use in contact with the tissues of human beings and animals without excessive toxicity, irritation, allergic response, or other problem or complication, commensurate with a reasonable benefit/risk ratio.

The pharmaceutical corn positions disclosed herein comprise a pharmaceutically-acceptable carrier, which, as used herein, includes, but are not limited to, any and all solvents, buffering agents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Various excipients for formulating pharmaceutical compositions and techniques for preparing the composition are known in the art (see Remington: The Science and Practice of Pharmacy, 21st Edition, A. R. Gennaro, Lippincott, Williams & Wilkins, Baltimore, Md., 2006; incorporated herein by reference). The use of a conventional excipient medium may be contemplated within the scope of the present invention, except insofar as any conventional excipient medium may be incompatible with a substance or its derivatives, such as by producing any undesirable biological effect or otherwise interacting in a deleterious manner with any other component(s) of the pharmaceutical corn position.

Exemplary preservatives may include, but are not limited to, antioxidants, chelating agents, antimicrobial preservatives, antifungal preservatives, alcohol preservatives, acidic preservatives, and/or other preservatives. Exemplary antioxidants include, but are not limited to, alpha tocopherol, ascorbic acid, ascorbyl palmitate, butylated hydroxyanisole, butylated hydroxytoluene, monothioglycerol, potassium metabi sulfite, propionic acid, propyl gallate, sodium ascorbate, sodium bisulfite, sodium metabi sulfite, and/or sodium sulfite. Exemplary chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and/or trisodium edetate. Exemplary antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and/or thimerosal. Exemplary antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and/or sorbic acid. Exemplary alcohol preservatives include, but are not limited to, ethanol, polyethylene glycol, phenol, phenolic compounds, bisphenol, chlorobutanol, hydroxybenzoate, and/or phenylethyl alcohol. Exemplary acidic preservatives include, but are not limited to, vitamin A, vitamin C, vitamin E, beta-carotene, citric acid, acetic acid, dehydroacetic acid, ascorbic acid, sorbic acid, and/or phytic acid. Other preservatives known in the art may also be used.

Exemplary buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, d-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and/or combinations thereof.

Formulations of the pharmaceutical compositions described herein may be prepared by any method known or hereafter developed in the art of pharmacology.

In various embodiments, the pharmaceutical composition of is a topical formulation.

The term “topical formulation” means a composition that may be placed for direct application to a skin surface and from which an effective amount of the biologically active component is released. Such formulations may include liquids, creams, ointments, gels, lotions, or any other dosage form suitable for topical application and the like. In some embodiments, such formulations may be applied to the skin in an unoccluded form with/without additional backing, structures or devices.

In a third aspect, the invention provides an organosilica nanoparticle or pharmaceutical composition disclosed herein for use as a medicament. In a fourth aspect, the invention provides an organosilica nanoparticle or pharmaceutical composition disclosed herein for use in the treatment of a disease, disorder, or condition, preferably cancer, more preferably skin cancer, most preferably melanoma.

In a fifth aspect, the invention provides a method for treating a disease, disorder, or condition in a subject, comprising the steps of:

(a) administering, preferably topically, to the subject a therapeutically effective amount of an organosilica nanoparticle or pharmaceutical composition disclosed herein; and

(b) exposing the subject to photoirradiation, thereby treating the disease, disorder, or condition at least partly by photodynamic therapy.

In various embodiments, the photoirradiation is by near-infrared light, preferably by 730-nm laser.

In various embodiments, the disease, disorder, or condition is selected from the group consisting of primary melanoma, metastasized melanoma, basal cell carcinoma, Bowen's disease, actinic keratosis, abnormal scarring (keloid and hypertrophic scars), atoptic dermatitis, warts, pre-malignant non-melanoma skin lesions, and cholangiocarcinoma.

In various embodiments, the disease, disorder, or condition is a skin cancer, preferably melanoma, and the organosilica nanoparticle or pharmaceutical composition is administered topically.

In various embodiments, the method comprises enhancing skin penetration of the organosilica nanoparticle using a microneedle patch. Microneedle patches can be used to pierce the stratum corneum and generate transient microchannels for enhanced transdermal transportation of the organosilica nanoparticles. Alternatively or additionally, any other means known in the art may also be used to facilitate the transdermal delivery of the organosilica nanoparticles. These include chemical, physical, and biological enhancers. Chemical enhancers are chemical compounds or formulation methodologies and help by perturbing the stratum corneum, increasing partition coefficient, or increasing solubility. Physical methods utilize equipment or device to physically generate routes for drugs to penetrate. They include electroporation, cavitational ultrasound, and microneedles, etc. The biological methods include the use of enzymes, synthetic lipid inhibitors, and other biologics that alter the metabolic balance and activity of the stratum corneum.

In various embodiments, the subject is a human or mammal.

The present invention is further illustrated by the following examples. However, it should be understood, that the invention is not limited to the exemplified embodiments.

### EXAMPLES

### Materials and methods

### Chemicals

Tetramethoxysilane (TMOS) was obtained from J&K Scientific, and 2-methoxy (polyethyleneoxy)-propyl) trimethoxysilane tech-90 was obtained from Gelest, Inc. Dabrafenib mesylate (GSK-2118436B) and trametinib (GSK-1120212, JTP-74057) were purchased from ActiveBioChem. All other chemicals were obtained from Sigma-Aldrich. Deionized water was used throughout the whole experiment.

### Characterization

Transmission electron microscopy (TEM) images were obtained on a TEM JEOL 1400 at 100 kV. Dynamic light scattering (DLS) was measured on a Malvern Zetasizer Z model. Brunauer-Emmett-Teller (BET) surface areas were obtained from the N₂ adsorption/desorption isotherms measured by the Quantachrome Instruments Autosorb-iQ (Boynton Beach, Fla. USA). UV-Vis spectroscopy was conducted on a Shimadzu UV-Vis-NIR 3600. Fluorescence spectra were measured on a Shimadzu RF 5301PC spectrometer. Flow cytometry was measured with a Fortessa X20 (3 laser) flow cytometer. 3-(4,5-Dimethylthiazol-2-yl)-2,5-diphenyltetrazolium bromide (MTT) assay was performed using a Tecan Infinite M200 microplate reader. X-ray photoelectron spectroscopy (XPS) was determined on Phoibos 100 SPECS using a monochromatic Mg X-ray radiation source. Elemental analysis was determined using a EuroEA CHNS-O Analyzer, EuroVector. Confocal laser scanning microscopy (CLSM) was imaged using a Carl Zeiss LSM800. Temperature measurements were carried out on an FLIR infrared camera thermometer. Porcine skin penetration experiments were conducted on an IVIS SpectrumCT Pre-clinical in Vivo Imaging System. Histology imaging was conducted on a Life Technologies EVOS microscope.

### Synthesis of Pc-Si

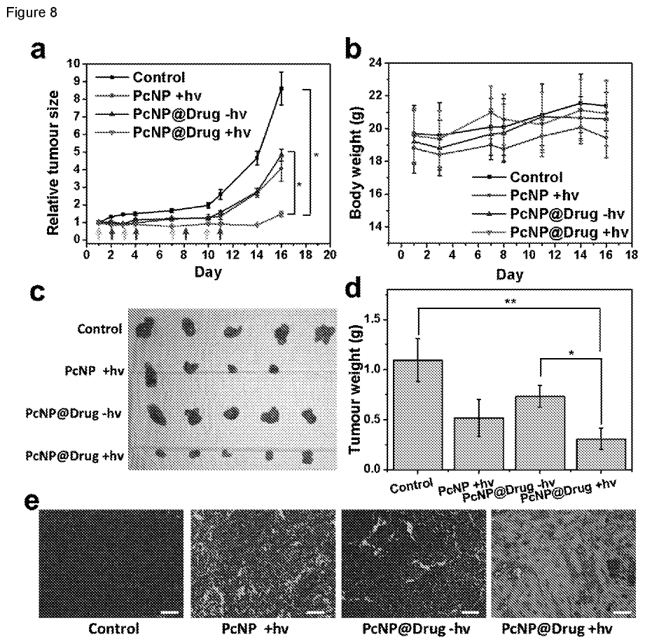

Briefly, Pc-4NH₂ (9.0 mg) was weighed and transferred to a three-necked flask. Anhydrous DMF (5 mL) was added to dissolve Pc-4NH₂. 3-(Triethoxysilyl)propyl isocyanate (13.8 μL) was dissolved in anhydrous DMF (0.1 mL) and then injected into the flask. The reaction was refluxed at 120° C. under nitrogen protection overnight. The resulting Pc-Si solution was directly used for the synthesis of PcNP (see FIG. 9).

### Synthesis of PcNP

CTAB (1.0 g) was dissolved in H₂O (120 mL). Triethanolamine (420 μL, 1:1 w/w in water) was added and the obtained solution was stirred vigorously at 80° C. for 30 minutes to form micelles. TMOS (160 μL) and Pc-Si (800 μL) were mixed evenly and then added dropwise into the CTAB solution under vigorous stirring. The reaction was allowed to proceed for 2 hours. Subsequently, the temperature was lowered to 50° C., and a 2-methoxy (polyethyleneoxy)-propyl) trimethoxysilane solution (400 μL, 1 g/mL in ethanol) was added dropwise. The reaction was left to stir overnight for complete reaction. The reaction mixture was dialyzed against a 10% v/v acetic acid/absolute ethanol solution for 3 days to remove unreacted silane precursors and CTAB, and then 2 days against DMSO to remove unreacted Pc-Si (MWCO 12,000). Lastly, it was dialyzed against water and freeze-dried.

### Synthesis of PcNP@Drug

Dabrafenib (2 mg/mL) and trametinib (2 mg/mL) in DMSO stock solutions were added to PcNP (1 mg) in DMSO to obtain a final ratio of 1 mg/mL drug loading solution. The drug loading was carried out with continuous stirring for 24 hours, after which the nanoparticles were washed repeatedly with ethanol and water to remove excess drug and DMSO. The product, PcNP@Drug, was collected by centrifugation at 9000 rpm for 45 minutes.

### Optimization of silane precursor ratio

The optimum ratio of Pc-Si to TMOS was determined by synthesizing a series of nanoparticles (A, B, C and D) with Pc-Si:TMOS molar ratios of 1:100, 270, 500 and 1000 respectively, where the total silane concentration was ensured to be same for all the samples. The singlet oxygen generation efficiency was then tested against 9,10-anthracenediyl-bis(methylene)dimalonic acid (ABDA). Solutions of A, B, C and D in water were separately mixed with ABDA solution, of which all solutions gave similar absorbance reading at 730 nm. Baseline was adjusted accordingly to negate the absorbance readings of the nanoparticles. The solutions were irradiated with 1 W/cm² 730 nm laser and the absorbance values were measured at various time intervals. The optimum Pc-Si to TMOS ratio determined by the one that gives the best quenching of ABDA was used for subsequent synthesis of PcNP.

### TEM images

PcNP or PcNP@Drug was dispersed in water, 10 μL of which was dropped on a carbon coated copper grid and allowed to dry in air for 24 hours before imaging using TEM.

### N₂ absorption/desorption and pore size analysis

Prior to the analysis, PcNP and PcNP@Drug were degassed at 180° C. in vacuum for 6 hours. The data were obtained by taking a range of P/P₀=0.05-0.2. The pore size distributions of PcNP were obtained using the DFT method.

### Determination of Drug Loading Content

DLC and EE were calculated against a calibration curve with 1:1 v/v mixture of dabrafenib and trametinib.

$$\begin{matrix}DLC = \frac{{drug}_{0} - {drug}_{supernatant}}{mass of PcNP @ Drug} \times 100 \% & eq ( 1 ) \\ EE = \frac{{drug}_{0} - {drug}_{supernatant}}{{drug}_{0}} \times 100 \% & eq ( 2 )\end{matrix}$$

where drug₀ stands for initial mass of drug in loading solution, and drugₛᵤₚₑᵣₙₐₜₐₙₜ stands for the mass of drug in supernatant after the loading.

### Photothermal Measurements

Solutions of PcNP at different concentrations (0, 0.1, 0.5, 1 mg/mL) were prepared in water. 730 nm 1 W/cm² laser was shone on the solutions for 10 minutes and the temperature rise was recorded with an infrared gun per every 30 seconds.

### Photostability Measurements

The PcNP solution and Pc-Si solution were prepared such that their optical density at 730 nm was similar. Both solutions were subjected to 730 nm, 1 W/cm² laser irradiation for 50 minutes, and corresponding absorbance spectra were recorded every few minutes. The optical density at 722 nm was then plotted against time.

### Cell Culture

Human BRAF^{V600E} melanoma cells (A375 and SKMEL-28), BRAF wild-type melanoma (B16F10), normal human epidermal keratinocytes (HEK), and normal human dermal fibroblasts

(HDF) were used. A375, HDF and B16F10 were cultured in Dulbecco's Modified Eagle's Medium (DMEM), and SKMEL-28 was cultured in Roswell Park Memorial Institute medium (RPMI) 1640. DMEM and RPMI 1640 were supplemented with 10% fetal bovine serum (FBS), penicillin (100 U mL⁻¹), and streptomycin (100 μg mL⁻¹). HEK was cultured in EpiGRO Human Keratinocyte Complete media supplemented with EpiGRO Human Keratinocyte Supplement Kit.

Cultures were grown under 5% CO₂ atmosphere at 37° C.

### Cellular Uptake Study

A375 cells were seeded in confocal dish. When the cells reached 70% confluence, PcNP was added to the cells at different time intervals at a concentration of 20 μg/mL. After the allocated duration for nanovehicle internalization, culture media were removed and the cells were washed thrice with PBS, fixed and analyzed by CLSM.

### Dosage Optimization

Cells were seeded in a 96-well plate and incubated overnight. Solutions made of different ratios and concentrations of dabrafenib to trametinib were prepared up to 10 μL. Similarly, 10 μL of each drug solution was added to 90 μL of fresh media. After incubation for 24 hours, the cell viability was tested via the MTT assay.

### Cumulative Drug Release

PcNP@Drug (2 mg) was dispersed in PBS (2 mL) at pH 7.4 and 5 each. The nanovehicle was stirred continuously for 48 hours. At certain time intervals, a portion of the solution was aliquoted out and centrifuged at 14800 rpm to obtain the supernatant, of which was put into a 96-well plate. Fresh PBS with the same volume was placed back into the release solution. The absorbance of the supernatant at different times were analyzed using a microplate reader, and the drug release kinetics was calculated against the calibration curve of dabrafenib and trametinib mixture.

### Cytotoxicity Studies

Cells were seeded at a density of 10⁵ cells per well in a 96-well plate and incubated overnight.

When cells reached ˜70% confluence, the media were removed and fresh media (90 μL) were added. PcNP solution in PBS was prepared in various concentrations up to 10 μL and added into the wells. Cells grown in 90 μL media and 10 μL PBS were used as controls. After the incubation in the dark for 48 hours, the culture media were removed, and a solution of MTT (10 μL, 5 mg/mL) with 90 μL fresh media was added into each well. After 4 hours, the media were replaced with 100 μL DMSO to dissolve the purple formazan crystals. The cell viabilities were calculated using the microplate reader at wavelength of 570 nm with reference at 690 nm.

For toxicity studies, cells were seeded in a 96-well plate and incubated overnight. PcNP and PcNP@Drug solutions in PBS were prepared in different concentrations. After 16 hours of incubation, the cells that require irradiation (PDT and PDT +drug combination) were irradiated with 730 nm 0.5 W/cm² for 8 minutes per well. Subsequently, the cells were incubated for another 16 hours before testing the cell viability via MTT measurements.

### Singlet oxygen quantum yield (ΦΔ) Determination

The singlet oxygen quantum yield of PcNP was measured by chemical means using DPBF as the singlet oxygen trap with reference to MB. The optical density of PcNP and MB solutions at 730 nm was first ensured to be similar. The baseline was adjusted to the absorbance spectrum of PcNP. The PcNP solution (750 μL) was added to the DPBF solution (50 μL, 2.5 mM), and the combined solution was irradiated with 730 nm, 1 W/cm² laser (0.1-5 W adjustable CW 730 nm laser, DL-730-1500, Model ADR-1805, Shanghai Solution Co. Ltd). The absorbance of DPBF was measured regularly over a 10-minute period. The same procedure was repeated for MB with DPBF. The absorbance values at 428 nm were recorded against time, and the curves were fitted using first order exponential fitting to obtain the time to decay (t) data. The singlet oxygen quantum yield of PcNP was calculated according to the formula Φ_{Δ(PcNP)}−Φ_{Δ(MB)}×(t_{MB}/t_{PcNP}), where Φ_{Δ(MB)} at 0.52 was obtained online.

### In Vitro Synergism Calculation

The quantitative analysis of the synergism between various treatment methods was conducted using the combination index (CI) theorem by Chou-Talalay (Chou, T.-C.; Talalay, P. Adv. Enzyme Regul. 1984, 22, 27-55). The effect of drug combinations could either be additive (CI=1), synergistic (CI<1), or antagonistic (CI>1). CI was calculated using CompuSyn software.

### In Vitro Oxidative Stress Detection

The in vitro oxidative stress was analyzed using the Image-IT® LIVE Green Reactive Oxygen Species Detection Kit from Thermo Fisher according to manufacturer's instructions. SKMEL-28 cells were seeded in a 12-well plate. PcNP was added at a final concentration of 25 μg/mL. After incubation for 12 hours, the cells were labelled with carboxy-H₂DCFDA for 10 minutes and washed with PBS. The cells corresponding to the PcNP+hv treatment were irradiated with 730 nm, 0.75 W/cm² laser for 20 minutes. A positive control using common ROS production inducer TBHP was added to a final concentration of 1 μM and incubated for 15 minutes. After the treatment, the cell nuclei were labeled with Hoechst 33342 (1 μM). Coverslips were then washed with PBS, fixed and mounted onto microscopy glass slides for imaging by CLSM. Carboxy-H₂DCFDA λ_{ex/em}: 488/529 nm, Hoechst 33342 λ_{ex/em}: 350/461 nm.

### Live/Dead Confocal Assay

Calcein AM and PI were obtained from Life Technologies. SKMEL-28 cells were seeded in p-Slide 4-Well Glass Bottom and left to adhere overnight. Prior to the addition of nanovehicle, the cells were starved by using serum-free media. PcNP and PcNP@Drug were added to a final concentration of 10 μg/mL. After the incubation for 4 hours, the cells corresponding to the PcNP@Drug+hv treatment and PcNP+hv treatment were irradiated with 730 nm, 0.5 W/cm² laser for 20 minutes each. Subsequently, the cells were left to incubate for additional 16 hours. To stain the cells, the culture media were removed, and a mixture of Calcein AM and PI was added into each well to stain the cells. Cells were incubated at 37° C. for 15 minutes. Then, the cells were rinsed with PBS twice and prepared for confocal imaging. Calcein λ_{ex/em}: 485/535 nm, PI λ_{ex/em}: 530/620 nm.

### Caspase 3 Activity

Caspase 3 was detected using a Caspase 3 Assay Kit (Colorimetric) from Abcam and was conducted according to manufacturer's instructions.

### 3D Tumor Spheroids

3D tumor spheroids were generated by using hanging drop method. 8000 A375 cells were dispersed in complete media (35 μL) and carefully pipetted on the lid of a cell culture dish in a spaced-out manner. The lid was carefully inverted over the dish that was filled with PBS (15 mL) to prevent the droplet evaporation. Spheroids were allowed to aggregate and grow for 2 weeks to achieve a diameter of 400 μm. After which, the treatment was started.

Every 2 days, droplet media (5 μL) were removed and replaced with fresh media. PcNP@Drug or PcNP was added accordingly, and incubated for 24 hours before irradiating with 730 nm laser every 2 days. Spheroid volume was calculated using the formula:

$$V = \frac{4}{3} π {r}^{3} .$$

where r is radius of spheroid.

For acid phosphatase assay, droplets of spheroids were carefully washed with ApH buffer before transferring 50 μL of spheroids to a 96-well plate. The ApH buffer was added to make up to 100 μL. p-Nitrophenyl phosphate (pNPP, 10 μL, 2 mg/mL) was added to each well, followed by the incubation at 37° C. for 3 hours. After which, NaOH solution (10 μL, 1 M) was added to quench the reaction. The absorbance was read at 405 nm with the reference at 630 nm. The ApH buffer was made using 0.1 M NaAc+0.1% Triton X-100.

### Topical Penetration of Porcine Skin

Fresh full-thickness porcine skin was obtained from a local wholesaler and cut into 1 cm by 1 cm pieces. The subcutaneous fats were gently stripped from the porcine skin. Any remaining skin was kept frozen at −20° C. and used as soon as possible. A pyramidal stainless steel microneedle patch consisting of 100 needles in a 10×10 array with a height of ˜500 μm, a tip radius of 5 μm, a pitch of ˜700 μm and a base width of ˜300 μm was obtained from Micropoint Technologies Pte Ltd (Singapore). To prove the efficacy of using microneedle patch on the penetration of skin, skins were split into 4 groups. PcNP solution at 20 mg/mL concentration was compared against free Pc (equivalent to the concentration of Pc in PcNP at 20 mg/mL). The penetration of PcNP was tested after two durations: 10 minutes and 1 hour. The penetration was conducted with and without the help of the microneedle patch. Briefly, if required, the skin was pierced with the microneedle patch under a force of about 4 N for 10 seconds before its removal. PcNP or free Pc solution (40 μL) was added to the skin to cover a circular area about 0.6 cm in diameter. After 10 minutes or 1 hour, the PcNP or free Pc solution was gently removed using a micropipette, and the skin was rinsed 3 times with PBS (50 μL) to remove any excess. PBS, PcNP solution (20 mg/mL), and free Pc solution as controls added onto the skins were not removed away. The fluorescence intensity of the adsorbed Pc was measured using an IVIS SpectrumCT Pre-clinical in Vivo Imaging System (Perkin Elmer), where λ_{ex/em} is 640/700-760 nm. The autofluorescence of the porcine skin was removed using a function in the instrument's software (Living Image). To calculate the percentage of PcNP that penetrated into the skin, the intensity reading for each skin was normalized against that of PBS and PcNP positive control. In the case of free Pc penetration, this percentage was calculated by normalizing the readings against that of PBS and free Pc positive control.

Fresh full-thickness porcine skin that was penetrated with 20 mg/mL PcNP for 10 minutes and 1 hour with or without microneedle patch were fixed in 4% paraformaldehyde, embedded in paraffin block, sectioned longitudinally, and mounted on glass slides that reduce autofluorescence. The sections were imaged on CLSM, λₑₓ: 488+561 nm, λₑₘ: 565-700 nm. The quantification was based on the corrected total cell fluorescence (CTCF) formula:

CTCF=Int den−(A×Fl_{background})

where Int den is integrated density, A is area of interest and Fl_{background} is the mean fluorescence of background, which were calculated using the software ImageJ.

### A375 Xenograft

Female homozygous CrTac:NCr-Foxn1nu NCr nude mice (4 weeks old) were used. A375 cells were cultured in T175 flasks and harvested once confluence was reached. Cells were mixed in Matrigel at a 1:1 v/v ratio. 4×106 cells (200 μL) were subcutaneously injected into the flank of each mouse. Five mice were used for each experimental group.

The care and use of laboratory animals were performed according to the approved protocols of the Institutional Animal Care and Use Committee (IACUC) at Nanyang Technological University, Singapore.

### In Vivo Efficacy of PcNP@Drug

When tumors were established in the mice, the treatment was started. The nanovehicle (PcNP or PcNP@Drug) treatment was conducted on days 1, 3, 7 and 10. The laser treatment was conducted the day after the nanovehicle treatment, i.e., days 2, 4, 8 and 11. This arrangement would allow sufficient time for the PcNP diffusion across boundaries of tumor tissues and maximize the nanocarrier internalization by melanoma cells. The nanovehicle treatment was comprised of anaesthetizing the mouse, followed by 30 seconds of the microneedle patch application, and addition of PcNP solution (40 μL of 50 mg/mL in 3% sodium carboxymethylcellulose). The procedure of laser treatment includes the anesthesia and irradiation by 730 nm, 2W laser at a height of 15 cm. For the drug group, the laser treatment was not carried out. Five mice were used per group and the tumor volume was measured regularly and calculated using the formula: volume=0.5×L×W², where L is the longest length of the tumor, and W is the width of the tumor. The relative tumor volume was calculated by the formula: relative tumor volume =(volume of tumor on day n/volume of tumor on day 1) x 100%.

The tumor growth inhibition ratios were calculated according to the formula % TGI=(V_{c}−Vₜ)/(V_{c}−V₀)×100%, where V_{c} stands for median volume of tumor in the control group at day 16 of the experiment, Vₜ stands for median volume of tumor in experimental group at day 16 of the experiment, and V₀ stands for median volume of tumor in control group at the start of the experiment.

### Histology Analysis of Tumors

On day 18 post-administration, the mice were euthanized by CO₂ inhalation. After which, the tumors were carefully extracted out, and fixed with 4% formaldehyde. The tumors were embedded in paraffin block, sectioned, stained with H&E and a TUNEL kit (Millopore S7101), and mounted on glass slides.

### Statistical Analysis

All data were expressed as mean±standard error of mean. Statistical difference between two sets of data was determined by one-way ANOVA and p<0.05 was considered to be statistically significant.

### Example 1: Microneedle-Assisted Topical Delivery of Photodynamically Active Mesoporous Formulation for Combination Therapy of Deep-Seated Melanoma

Malignant melanoma has high prevalence, particularly in the Caucasian population, with over a million cases detected each year. It takes up 4% of skin cancer incidence, but accounts for 79% of skin cancer mortalities. It is resistant to radiotherapy and chemotherapy, with the latter showing serious side effects due to nonspecific targeting. Surgical resection is ineffective in 20% of all cases too. Recently, targeted therapy has been employed to improve the overall survival rate of melanoma. Over 60% of melanoma relates to the BRAF mutation, of which 90% are of the subtype BRAF^{V600E}. BRAF^{V600E} is due to hyperactive mitogen-activated protein kinase (MAPK) pathway, resulting in over-stimulated cell transformation and proliferation.

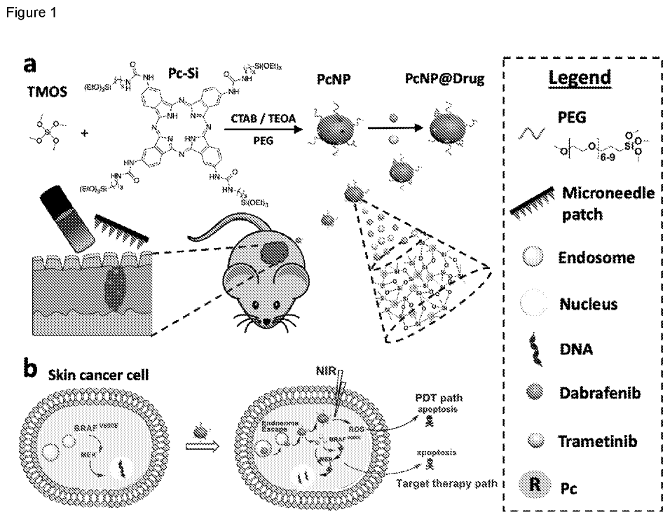

Described herein is a novel and inventive technology that addresses the abovementioned limitations (FIG. 1). Specifically, the inventors developed a drug-containing mesoporous organosilica nanocarrier that is pre-conjugated with a photosensitizer (i.e. phthalocyanine). As PDT does not require the release of photosensitizers, the covalent linkage of photosensitizers in the silica matrix would allow sufficient loading of photosensitizers and yet prevent their aggregation-induced quenching, thus increasing the quantum yield of photosensitizers in the system. The porous nanostructure could also facilitate the co-loading of therapeutic drugs. As a proof of concept, the inventors co-encapsulated two FDA-approved drugs, i.e., dabrafenib and trametinib, in organosilica nanoparticles for the combination treatment of mutant melanoma.

As disclosed herein, microneedle patches were used due to their simplicity and commercial availability. Using them do not require specific training and licensing, and they are cheap as compared to other physical enhancement techniques (e.g. microdermabrasion). Microneedle patches have three-dimensional microstructures with microscale length. The treatment procedure is as follows. Firstly, the microneedle patches were used to pierce the stratum corneum and generate transient microchannels. Secondly, drug-loaded nanoparticles were topically applied. These nanoparticles can enter the skin through the microchannels and diffuse within the skin layers. Lastly, PDT was performed. After the treatment, the tumor was observed to shrink in the mouse models within 16 days.

The combination of dabrafenib and trametinib has been approved for the treatment of BRAF^{V600E} unresectable melanoma. Dabrafenib and trametinib inhibit BRAF (a protein kinase activator) and the downstream MEK pathway, respectively. In clinics, this combination is given in high dosages orally, but has low bioavailability and a range of potentially fatal side effects. Using the nanocarrier topically to enhance the accumulation of both drugs at the melanoma site would significantly reduce the burden on the body.

Phthalocyanine (Pc) functionalized with four silicate units (Pc-Si) was first synthesized using a similar method reported in literature (FIG. 9) (Tham, H. P., et al. Chem. Commun. 2016,52, 8854-8857; Lindig, B. A., et al. J. Am. Chem. Soc. 1980, 102, 5590-5593). Pc can be excited by far-red light that is able to penetrate into the dermis of the skin, where melanoma infiltrates. Pc-bonded mesoporous organosilica (PcNP) was then synthesized using Pc-Si via silane co-condensation and hydrolysis. Hexadecyltrimethylammonium bromide (CTAB) was used as the structure-directing agent to form micelles in the presence of triethanolamine (TEOA), a basic catalyst (Mizoshita, N.; Tani, T.; Inagaki, S. Chem. Soc. Rev. 2011, 40, 789-800.). Tetramethyl orthosilicate (TMOS) was chosen as the inorganic silica source over commonly used tetraethyl orthosilicate (TEOS) because of its higher water solubility. This method results in a hastened but controlled completion of the hydrolysis process, allowing the formation of uniform small particles suitable for topical delivery (Yamada, H., et al. Chem. Mater. 2012, 24, 1462-1471). The two precursors, TMOS and Pc-Si, were added dropwise under vigorous stirring. Subsequently, 2-methoxy (polyethyleneoxy)-propyl) trimethoxysilane (PEG) was added to quench particle growth, and provide hydrophilicity to the resultant PcNP. The PcNP was then purified via dialysis. Small inhibitor drugs, dabrafenib and trametinib, were loaded into the PcNP pores to obtain drug-loaded PcNP@Drug (FIG. 1). When mice were treated with PcNP@Drug delivered by a microneedle patch, PcNP@Drug was able to produce reactive oxygen species (ROS) in vivo under NIR light irradiation. In addition, the release of the drugs could inhibit mutant BRAF and the subsequent MEK pathway of cancer cells.

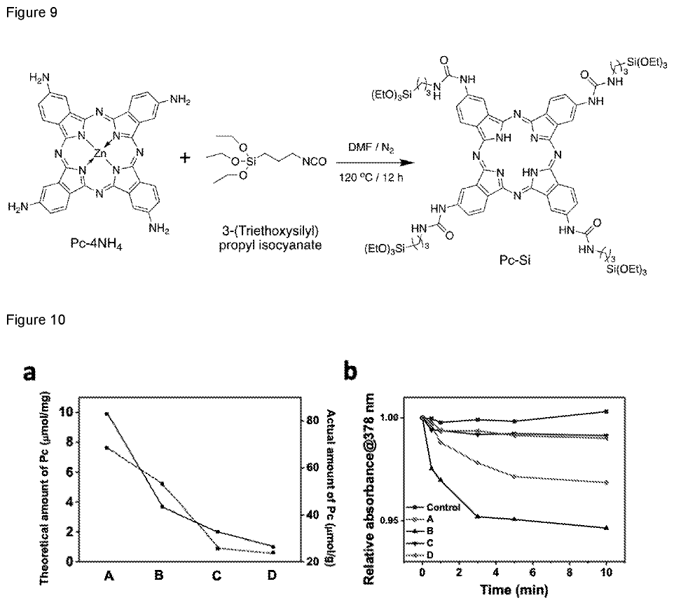

It is well known that, when some photosensitizers aggregate, strong π-π stacking may cause their ROS-generating ability to be hindered due to quenching of their excited state (Ali, H.; van Lier, J. E. Chem. Rev. 1999, 99, 2379-2450). In order to find out the optimum concentration of Pc to be included in each nanoparticle, different TMOS to Pc ratios (100, 270, 500, and 1000 : 1) were used and denoted as A, B, C and D, respectively. It was observed that when the TMOS:Pc ratio decreased further, nanoparticles failed to form on account of large amount of Pc-Si used. After the nanoparticles were synthesized and purified, the Pc content was analyzed by elemental analysis (EA) and calculated according to the nitrogen weight percentage (FIG. 10a and Table 1).

TABLE 1
Tabulation of the theoretical Pc and actual
loaded Pc and their corresponding
efficiency. Efficiency = Actual Pc/Theoretical Pc × 100%.

| | | Theoretical Pc | Actual Pc | Efficiency |
|---|---|---|---|---|
| Sample | TMOS:Pc | (μmol/mg) | (μmol/g) | (%) |
| A | 100:1 | 9.90 | 68.5 | 0.69 |
| B | 270:1 | 3.69 | 53.2 | 1.44 |
| C | 500:1 | 1.99 | 25.7 | 1.29 |
| D | 1000:1 | 1.00 | 24.2 | 2.42 |

To determine the optimum concentration of Pc in PcNP, the inventors employed a chemical ROS trapping method using 9,10-anthracenediyl-bis(methylene) dimalonic acid (ABDA) as the trap agent. The absorbance of ABDA decreases as it reacts with ROS irreversibly to form an endoperoxide (FIG. 10b). It was observed that nanoparticles C and D quenched the absorbance of ABDA slightly, while nanoparticle B gave the most obvious quenching effect. Nanoparticle A, however, showed only intermediate quenching effect due to the abovementioned aggregation-induced quenching. Through this experiment, it was proven that the TMOS:Pc ratio used in the synthesis of nanoparticle B was the optimum.

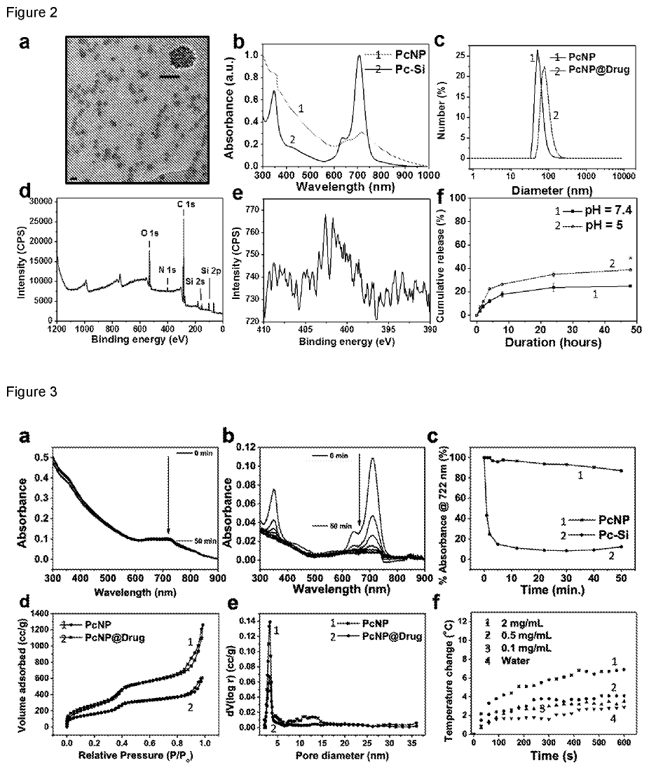

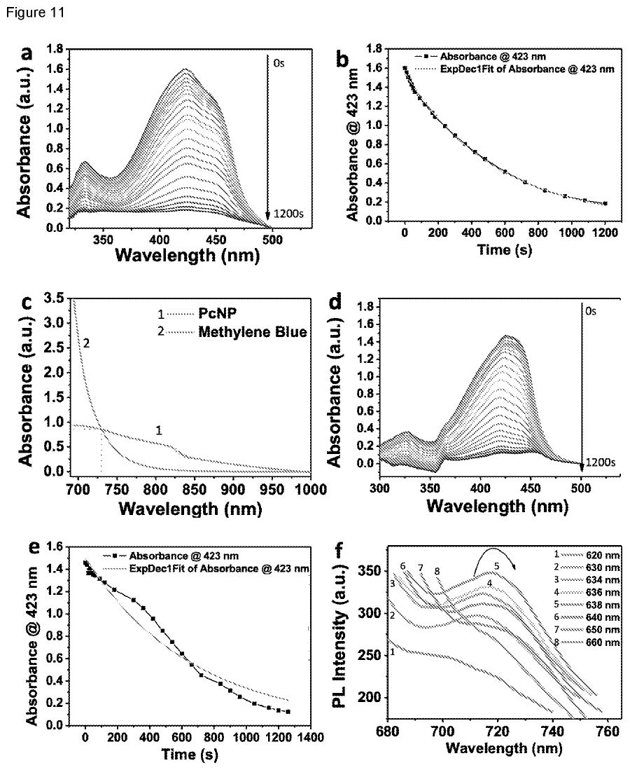

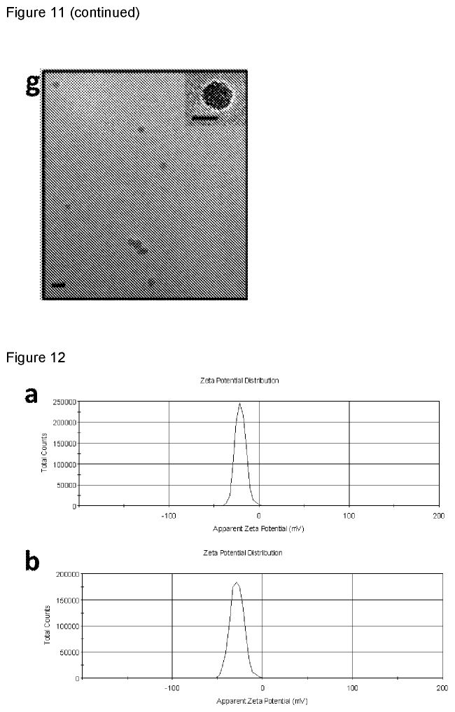

The synthesized PcNP was monodisperse as observed by transmission electron microscopy (TEM, FIG. 2a), with a diameter of 33±4 nm (n=30). After drug loading, PcNP@Drug showed a diameter of 34±5 nm (n=30, FIG. 11), meaning that there was no visible aggregation after drug loading. The hydrodynamic diameter was 50 nm and 78 nm for PcNP and PcNP@Drug respectively, as determined by dynamic light scattering (DLS, FIG. 2c). This slight increase in hydrodynamic diameter could be attributed to the change in the light refraction index of PcNP after the drug loading. The polydispersity index (PDI) was measured to be 0.161±0.004 for PcNP@Drug, indicating that PcNP was highly monodispersed after the drug loading. The zeta potential of PcNP was -21.3 ±0.8 mV, and after drug loading for PcNP@Drug, it was 28.7±0.4 mV (FIG. 12). Highly negative zeta potential of the nanoparticles confers great electrostatic stabilization and dispersability in solution. The absorption spectra (FIG. 2b) indicate that Pc was successfully incorporated into the framework, in which the spectrum of silylated pthalocyanine (Pc-Si) in DMSO displayed a Q-band at 707 nm, whereas that of PcNP in water red-shifted to 718 nm. The shoulder peak of Pc-Si at 636 nm similarly red-shifted to 649 nm in PcNP. This redshift behavior is a typical indication of the interaction between Si groups of Pc-Si and TMOS, due to the changes in the medium environment and the conformation of Pc when conjugated with silica in a 3D structure. The X-ray photoelectron spectroscopy (XPS, FIG. 2d) indicates the presence of oxygen at 531.6 eV, originated from TMOS. Relatively high carbon 1s peak at 284.5 eV was due to the carbon in the framework. The silicon 2s and 2p peaks at 153.6 eV and 67.5 eV were contributed by silicon in TMOS and Pc-Si, respectively. In addition, the nitrogen 1s peak from Pc was observed at 401.7 eV (FIG. 2d,e), indicating that Pc was successfully incorporated into the nanoparticles.

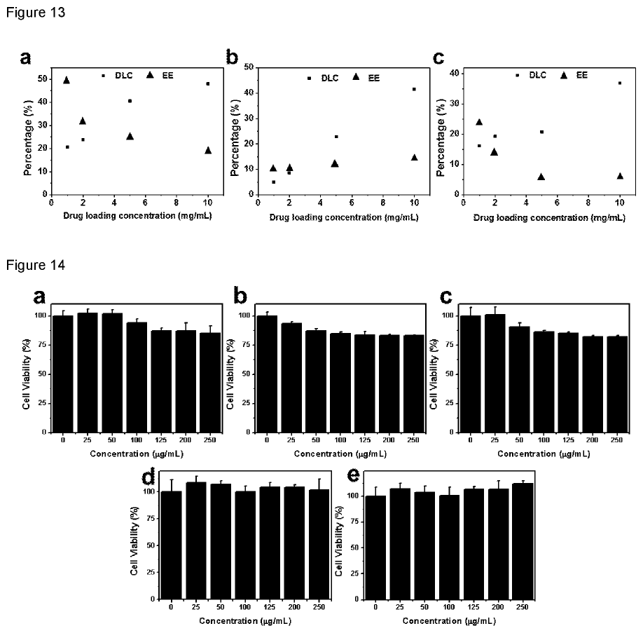

The drug loading capacity (DLC) and encapsulation efficiency (EE) of PcNP@Drug were determined by the calculations against the calibration curve of Pc-Si. Various drug loading concentrations (1, 2, 5, 10 mg/mL) of dabrafenib, trametinib and their combination were tested, and the corresponding DLC and EE values were plotted (FIG. 13). Generally, the DLC increases with increasing drug loading concentration, while the EE decreases. The loading of trametinib was lower than dabrafenib due to its lower solubility. At 10 mg/mL of dabrafenib +trametinib loading concentration, the DLC was 36.9±7.8% and the EE was 11.9±3.5%. At 1 mg/mL, the DLC and EE were 16.2±1.1% and 41.0±1.6%, respectively. For subsequent experiments, 1 mg/mL concentration was used as it was proven to be sufficient for cellular experiments.

A nitrogen weight percentage of 1.19 wt% was derived from elemental analysis results, and the corresponding Pc content in PcNP was calculated to be 53.2 _{N}mol/mg. The inventors then tested the cumulative drug release kinetics of the inhibitors inside PcNP@Drug at different pH levels (FIG. 2f). At pH 7.4, PcNP@Drug released 3.5% of its payload in the first hour before tapering off to a total of 24.9% after 48 hours. At pH 5, PcNP@Drug released 5.9% in the first hour and a total of 38.9% after 48 hours. This drug release amount is sufficient for the therapy, as demonstrated in the following studies. This sustained release means that the loaded drugs are not prematurely released in the epidermis of the skin, and PcNP@Drug would be accumulated to a large extent at the malignant sites before the drugs are released to their maximum. Furthermore, the increased drug release at acidic pH is beneficial, as endosome escape can be hastened.

The singlet oxygen generation quantum yield (ΦΔ) of PcNP was calculated by indirect chemical means using 1,3-diphenylisobenzofuran (DPBF, FIG. 11a,d). The optical density at 730 nm for PcNP and methylene blue (MB) was ensured to be similar (FIG. 11c). The ΦΔ of PcNP was calculated to be 0.42, considerably high for a synthesized organic photosensitizer in aqueous solution (FIG. 11b,e). For comparison, the ΦΔ of Pc-4NH₂ was determined to be 0.43 and ΦΔ of Pc-Si was 0.40. The photostability of PcNP was then investigated. The absorbance curve of PcNP was barely quenched after 50 minutes of irradiation (FIG. 3a). In contrast, the curve for Pc-Si was almost completely quenched, with a huge decrease of 85.3% within the first 5 minutes of irradiation (FIG. 3b). The relative absorbance at 722 nm throughout the course of laser irradiation was plotted (FIG. 3c), proving that the silica network could protect photosensitizers from photodegradation when Pc was incorporated into the framework. The high stability is because that a greater number of anchoring sites for Pc reinforces its structure inside the silica nanoparticles. Singlet oxygen and/or other ROS tend to attack the methine chain of Pc, and this reinforcement makes the chain more robust, hence stabilizing it during the photoirradiation. Another explanation for the increased photostability is that the Pc molecule is protected from the external environment by the silica matrix, preventing unexpected quenching by the external surface adsorbates or redox-active molecules.

A type IV isotherm was obtained by N₂ adsorption/desorption analysis for both PcNP and PcNP@Drug, displaying a pore-condensation step located around p/po =0.35-0.45 and another one at higher relative pressures of p/po =0.8-1.0, indicative of uniform mesoporosity. Type H1 hysteresis observed at around p/po =0.8-1.0 is evident of textural mesoporous characteristics of PcNP (FIG. 3d). The Brunauer-Emmett-Teller (BET) surface areas were estimated to be 1036 and 597 m²/g for PcNP and PcNP@Drug, respectively. The high surface areas of the nanoparticles, even after drug loading, could be attributed to their small particle size. The pore size revealed narrow distributions, peaking at 3.2 nm for both PcNP and PcNP@Drug (FIG. 3e). The sorption isotherms retained a similar shape after drug loading, implying no changes in the pore structure during drug loading. The pore volumes of PcNP and PcNP@Drug were 1.763 and 0.851 cm³/g respectively, where the pore volume of PcNP@Drug was lower as they were occupied with drugs. The reduction in the intensity of dv/(log r) from 0.14 to 0.06 after the drug loading also indicates that drugs were successfully loaded into the mesopores of PcNP.

The photothermal behavior of PcNP was then investigated. PcNP in aqueous solutions at various concentrations was tested and corresponding temperature changes were recorded (FIG. 3f). After 10 minutes of irradiation, the temperature of water was enhanced by 2.9° C., and that of 0.1, 0.5 and 2 mg/mL PcNP solutions was only increased by 3.5, 4.1 and 6.9° C. respectively. For cellular experiments, a low weight concentration about 0.15 mg/mL PcNP was used and this concentration did not give any appreciable temperature rise upon the irradiation. This observation confirms the rather poor photothermal performance of PcNP and also means that its photodynamic capability is high.

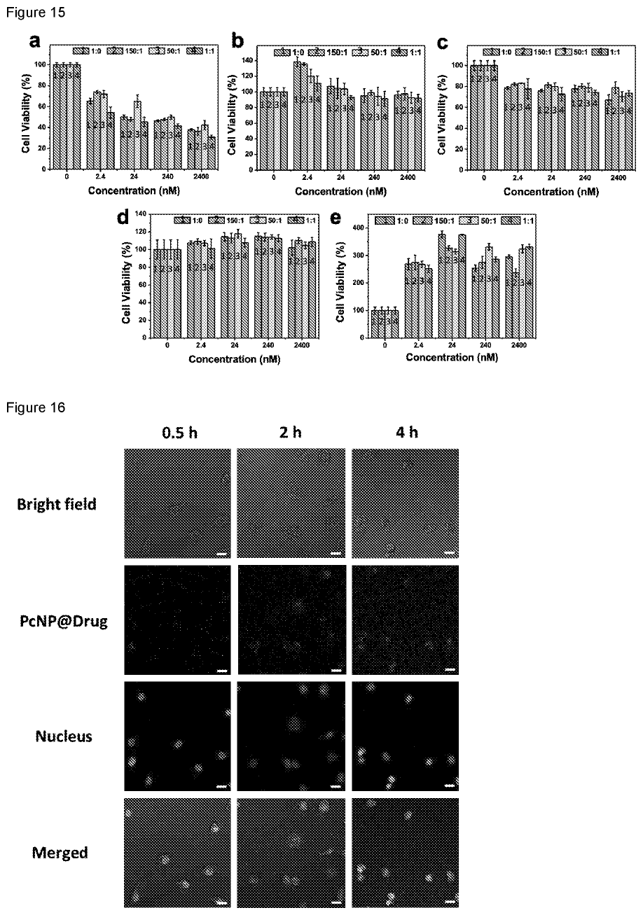

The cytotoxicity of PcNP in the absence of drugs and light was tested on various cell lines (FIG. 14). After 48 hours of incubation across a range of concentrations, the cells survived well, proving low dark cytotoxicity of PcNP even at high concentrations. Clinically, dabrafenib and trametinib are administered in a 150:1 ratio daily. As this ratio was hard to control, the inventors explored different ratios for the best therapeutic efficacy in vitro. The drugs were effective against BRAF^{V600E} mutant cells (SKMEL-28 and A375) (FIG. 15a,c), and much less effective to BRAF wild-type cell line B16F10 (FIG. 15b). Healthy cells (HDF and HEK) showed no obvious toxicity (FIG. 15d,e). The specificity of dabrafenib and trametinib only to BRAFV^{600E} mutant cells is indicated. For all cell lines, the therapeutic efficacy was not significantly different when the ratio of dabrafenib to trametinib was varied.

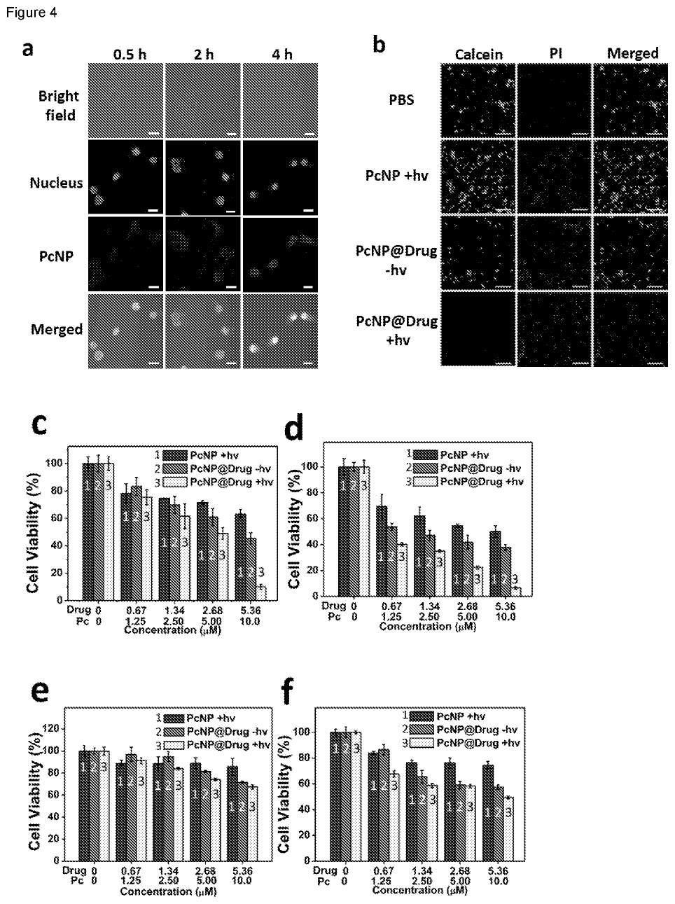

Time-dependent internalization of PcNP and PcNP@Drug was conducted on A375 cells (FIGS. 3a and 16). The nanoparticles were internalized into the cytoplasm of the cells as quick as within 0.5 hour, and showed a gradual increase in uptake with time, as evidenced by increasingly stronger red fluorescence in the PcNP or PcNP@Drug channel. Within 4 hours, the nanovehicles were observed to translocate into the nuclear region. Previous studies have shown that silica- based nanovehicles of similar charge and size could undergo clathrin-mediated endocytosis, and are capable of entering the nucleus. The efficacy of the PcNP@Drug system was then examined in a live/dead cellular assay using confocal laser scanning microscopy (CLSM, FIG. 4b). PBS control showed no visible presence of dead cells.

PcNP with irradiation (PcNP+hv) and PcNP@Drug without irradiation (PcNP@Drug-hv) showed some red fluorescence in the propidium iodide (PI) channel, depicting some but not total cell death, as seen in the merged channel. However, in the case of PcNP@Drug with irradiation (PcNP@Drug+hv), there was no visible green fluorescence but a strong red fluorescence in the merged channel, suggesting complete cell death.

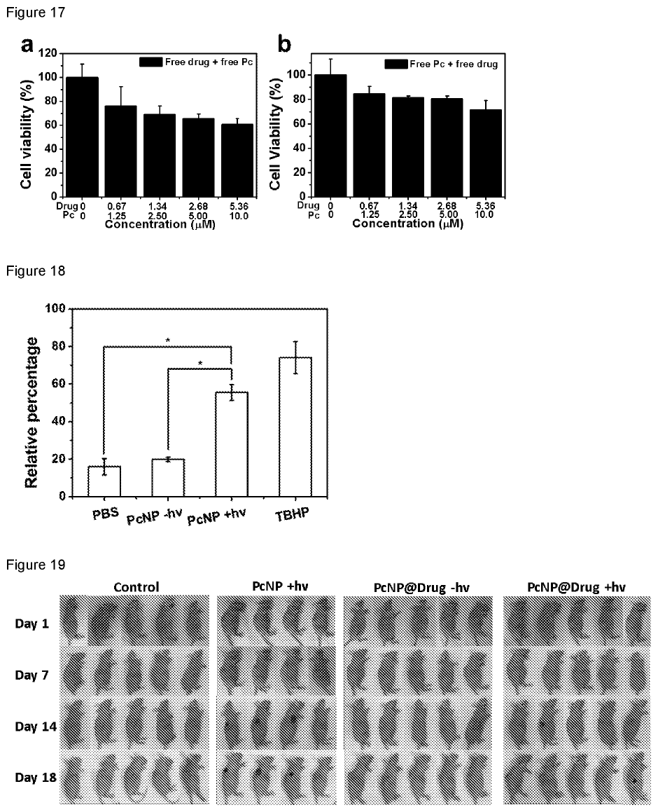

The therapeutic effect on different cell lines by PcNP@Drug was tested using A375, SKMEL-28, B16F10 and HDF cell lines, of which A375 and SKMEL-28 are the targeted BRAF mutant cell lines, and B16F10 is wild-type melanoma. Combinational therapeutic effect of PcNP@Drug+hv was compared against single treatment of PDT (PcNP+hv), targeted therapy (PcNP@Drug-hv) alone, and physical combination of free Pc and free drugs. In both SKMEL-28 and A375 cell lines at all concentrations, the combination treatment was able to kill more cells than single treatment (FIG. 4c,d) and physical combination of free Pc and free drugs (FIG. 17a). This therapeutic effect was more pronounced at higher concentrations, where the cell viability decreased to 10.0% and 6.2% for A375 and SKMEL-28 cells respectively, corresponding to 5.36 pM drug and 10.0 pM Pc. The effect of single PcNP+hv treatment was limited as observed by the plateau effect even when the concentration was increased. HDF cells were rather resistant to the treatment, only showing a slight decrease to 67.5% cell viability at the highest concentration. This result proves that topical treatment of PcNP@Drug does not affect the dermis layer of the skin as compared to the targeted melanoma cells, if it should penetrate deeply. The combination treatment showed a synergistic effect (combination index =0.79 for A375, and 0.44 for SKMEL-28 cell lines) as calculated by the Chou-Talalay method, indicating an efficient therapy for the targeted cells. The synergistic effect was detected in A375 with equivalent 1.25 μM Pc and 0.67 μM drug, whereas it was efficient against SKMEL-28 under all concentrations used. It was also observed that the ROS generation from PcNP+hv in the combination treatment was toxic toward HEK cells (data not shown). In order to maximize the therapeutic efficacy, microneedles were used to increase the penetration into the malignant region as discussed later.

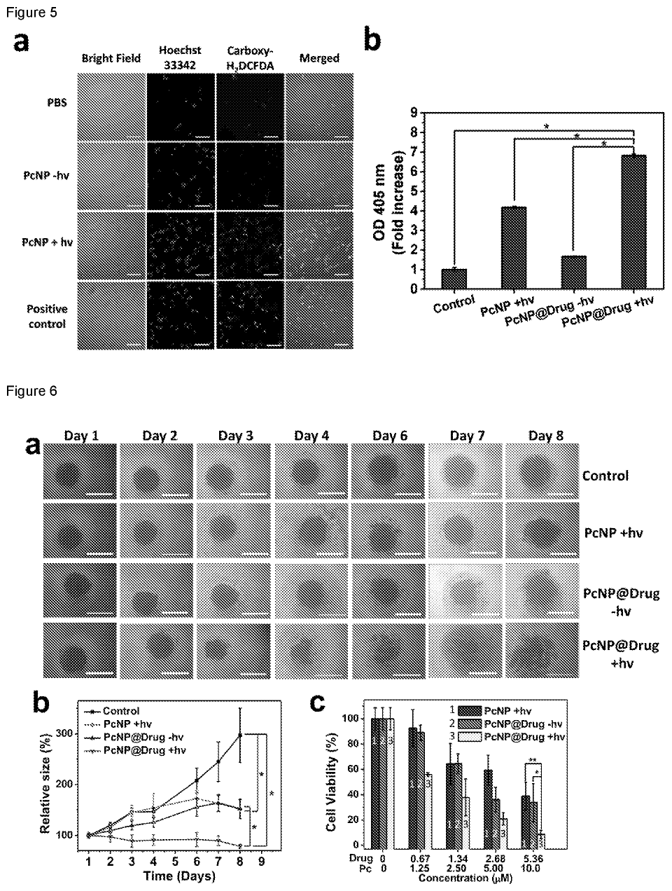

The in vitro oxidative stress detection assay using PcNP was conducted to prove that Pc in PcNP was responsible for producing oxidative stress only in the presence of light (FIG. 5a). In the PBS control and PcNP in the absence of light (PcNP-hv), no observable green fluorescence was detected, suggesting low dark toxicity of PcNP. In the presence of NIR light (PcNP+hv), green fluorescence was observed in the cytosol, merging well with the location of each cell. This observation was similar to the positive control experiment conducted using tert-Butyl hydroperoxide (TBHP) that chemically induces oxidative stress in vitro. Quantitative assessment (FIG. 18) shows that, in the PBS and PcNP-hv groups, only about 15% and 19% cells exhibited visible green fluorescence in the green channel, respectively. Significantly, an obvious increment to 55% cells became oxidatively stressed after the irradiation, and about 74% in TBHP-treated cells, confirming that Pc in PcNP can only be activated in the presence of light and is responsible for producing the ROS.

The activity of crucial caspase 3 protein was then determined to test the apoptotic activity of cells upon different treatments (FIG. 5b). Under PcNP+hv treatment, the cells displayed a 4.1-fold increase in caspase 3 protease. When treated with PcNP@Drug-hv, this increase was 1.6-fold. Upon PcNP@Drug+hv treatment, the caspase 3 protease increased by 6.8-fold, further indicating that the combination treatment is effective and the apoptosis is a possible mechanism for the cell death.

In addition, therapeutic efficacy of the nanovehicle on 3D tumor spheroids was investigated.

Digital photographs indicate that the control group of spheroids without treatment increased in size steadily from 470 μm to 670 μm (FIG. 6a). The spheroids receiving single treatment of either PcNP+hv or PcNP@Drug-hv grew slightly in size. Interestingly, the spheroids receiving the combination PcNP@Drug+hv treatment had the best performance, with their size shrinkage from day 3 and disaggregation from day 4 onwards. The diameters of the spheroids shrank from an average of 502 μm to 452 μm over 8 days. The corresponding volumes of the spheroids were calculated and their relative sizes were plotted (FIG. 6b). At the end of the treatment, the combinational PcNP@Drug+hv was significantly more effective than single treatments and control group. The cell viabilities of the spheroids were also analyzed using an acid phosphatase assay, as reported in several studies. Across all concentrations, the combinational

PcNP@Drug+hv treatment was shown to have much better cell-killing efficacy than PcNP+hv (PDT), PcNP@Drug-hv (targeted therapy) alone, or the physical mixture of free Pc and free drugs (FIG. 17b). At the highest concentration, the cell viability of the spheroids receiving single treatment dropped to 39% and 33% for PcNP+hv and PcNP@Drug-hv, respectively. On the other hand, the cell viability of the combination treatment was much lower at 8% (FIG. 6c).

The effectiveness of microneedles in improving the skin penetration of PcNP was tested on porcine skin, which is the most suitable model for human skin. The penetration of PcNP was tested against free Pc, with and without the help of microneedle patches.

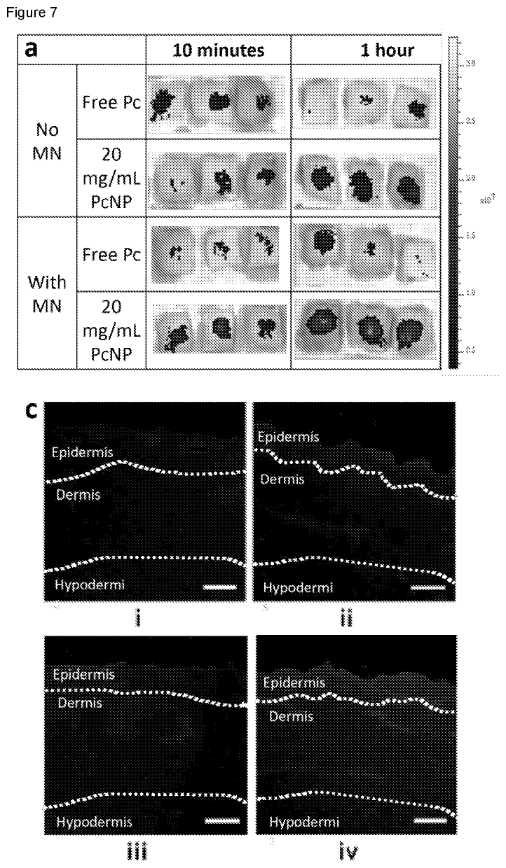

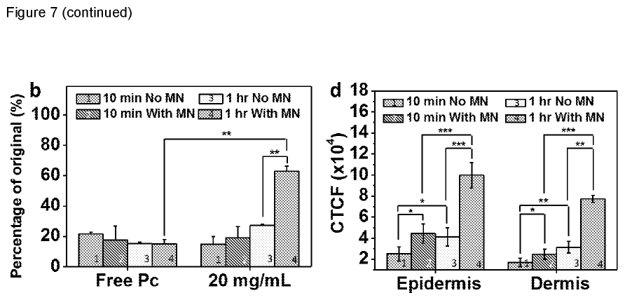

After the topical delivery of PcNP or Pc, the inventors carried out IVIS ex vivo imaging (FIG. 7a) and histological analysis of the skin samples (FIG. 7c). At 10 min, there was no significant difference of nanoparticle signals between the untreated and microneedle-treated skin samples (FIG. 7a,b). One hour later, however, the signal on microneedle-treated samples increased dramatically. The amount penetrated was 27.2% and 63.1% without and with microneedle, respectively. The inventors did not observe significant changes of nanoparticle penetration when there was no microneedle assistance. This observation indicates that it took 1 hour for PcNP to penetrate and distribute in the skin layers. Interestingly, there was minimal skin penetration of free Pc, regardless of the microneedle treatment. This result should be due to the hydrophobicity of the drug, which cannot diffuse through the skin.

We then proceeded to examine the histological samples of the treated skin sections using confocal imaging (FIG. 7c). As shown in FIG. 7c(i), there was barely any signal from nanoparticles in skin after the 10-min topical treatment of the formulation. This signal in the epidermis layer increased a bit with time (FIG. 7c(iii)). However, the signal in the dermis layer remained visually unchanged, indicating that PcNP was unable to get into the dermis layer even after longer duration of time. When the skin was pre-treated with microneedles, the nanoparticle signal in the epidermis layer was already strong only after 10 min (FIG. 7c(ii)). After 60 min, enhanced fluorescence signal in both epidermis and dermis layers could be seen. More interestingly, the signal distributes evenly throughout each layer, suggesting that PcNP penetrates into the skin through the diffusion.

The corrected total cell fluorescence (CTCF) in the porcine skin was quantified by taking the average of 4 regions in both the epidermis and dermis of the skin (FIG. 7d). Initially at 10 min and without microneedle pretreatment, there was not much signal throughout the skin. But with time, a 64% increase in the epidermis and an 85% increase in the dermis were detected. When the skin was pre-treated with microneedle, the fluorescence signal increased in both the epidermis (78%) and dermis (46%) after 10 min. After 1 hour of application, the increase was more prominent in both the epidermis (142%) and dermis (152%). The signals in the epidermis and dermis also increased by 112% and 368% when pre-treated with microneedle. These observations positively demonstrate the benefit of using microneedles in aiding the penetration of the PcNP nanovehicle.

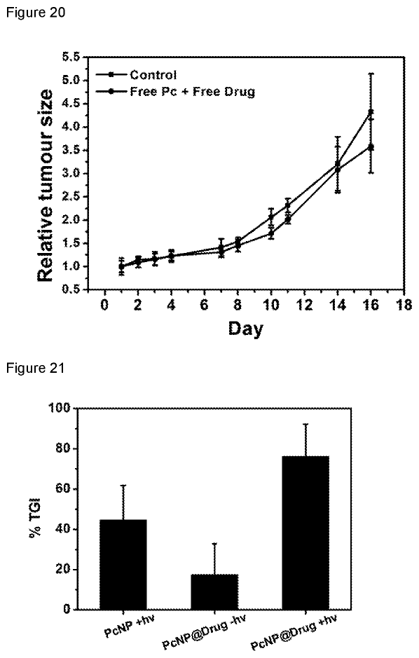

Finally, using a xenografted tumor mouse model, the inventors examined the anti-tumor efficacy of the drug-loaded nanovehicle (PcNP@Drug) through the combination treatment comprising of PDT, targeted therapy and microneedles (FIG. 19). The tumor model was established with subcutaneous injection of A375 cells into flanks of 4-week-old homozygous female CrTac:NCr-Foxnlnu mice. When tumors reached a size of about 5 mm in diameter, microneedle-assisted nanovehicle (PcNP or PcNP@Drug) treatment was conducted twice weekly for two consecutive weeks (as indicated by green arrow in FIG. 8a), followed by NIR laser treatment (as indicated by red arrow in FIG. 8a). As shown in the tumor growth curve (FIG. 8a), the control group exhibited exponential growth of tumors, while either PcNP+hv (PDT) or PcNP@Drug-hv (targeted therapy) treatment alone showed modest inhibition of tumor growth. This is also similar for the mice receiving physical mixture of free drug and free Pc without the microneedle aid (FIG. 20). In comparison, the PcNP@Drug+hv combination treatment led to significant tumor regression as compared to PBS control or PcNP@Drug-hv alone (P<0.05). In addition, the PcNP+hv and PcNP@Drug-hv groups gave tumor growth inhibition (TGI) values of 44.4% and 17.2% respectively, whereas the PcNP@Drug+hv combination group showed a TGI value of 76.0% (FIG. 21). Since a TGI>50% is considered meaningful,⁵⁰ these data conclude excellent anti-tumor efficacy of the PcNP@Drug nanovehicle.

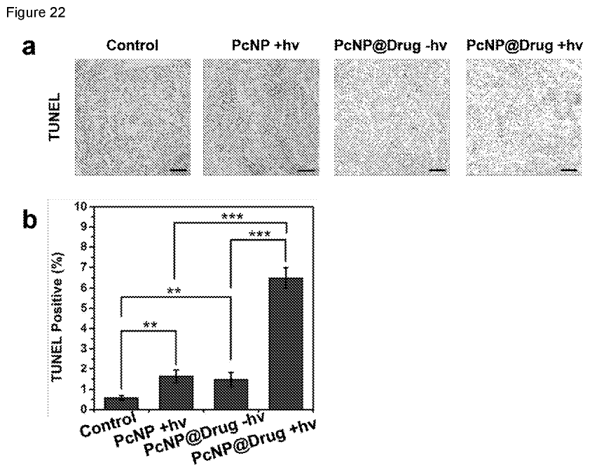

After the course of the study, the mice were sacrificed and tumors were excised and photographed in FIG. 8c. The tumors from the PcNP@Drug+hv group were obviously smaller in size than that of other groups. The tumor weights in the PcNP@Drug+hv group were also significantly lighter than other groups (FIG. 8d). In the control group, it was evident that tumor sizes enlarged over the whole treatment duration, whereas the tumor sizes significantly shrunk for the PcNP@Drug+hv group. Hematoxylin and eosin (H&E) staining of tumor slices obtained from different treatment groups of mice showed severe destruction of cancerous cells in the combination treatment group (PcNP@Drug+hv). The images obtained from single therapy groups (PcNP+hv or PcNP@Drug) displayed only slight damage. This observation confirms the tumor inhibition data and further demonstrates superior efficacy of the treatment through the combination of PDT and inhibitors (FIG. 8e). In addition, the terminal deoxynucleotidyl transferase dUTP nick end labeling (TUNEL) staining of tissue slices from different treatment groups confirmed that the ratio of apoptotic cells significantly increased in the PcNP@Drug+hv group as compared to that of control or single therapy groups (FIG. 22). This type of combination treatment was proven to be safe for topical in vivo applications, where the weights of the mice were well maintained over the whole treatment period (FIG. 8b).

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. Other embodiments are within the following claims. In addition, where features or aspects of the invention are described in terms of Markush groups, those skilled in the art will recognize that the invention is also thereby described in terms of any individual member or subgroup of members of the Markush group.

One skilled in the art would readily appreciate that the present invention is well adapted to carry out the objects and obtain the ends and advantages mentioned, as well as those inherent therein. Further, it will be readily apparent to one skilled in the art that varying substitutions and modifications may be made to the invention disclosed herein without departing from the scope and spirit of the invention. The compositions, methods, procedures, treatments, molecules and specific compounds described herein are presently representative of preferred embodiments are exemplary and are not intended as limitations on the scope of the invention. Changes therein and other uses will occur to those skilled in the art which are encompassed within the spirit of the invention are defined by the scope of the claims. The listing or discussion of a previously published document in this specification should not necessarily be taken as an acknowledgement that the document is part of the state of the art or is common general knowledge.

The invention illustratively described herein may suitably be practiced in the absence of any element or elements, limitation or limitations, not specifically disclosed herein. Thus, for example, the terms “comprising”, “including,” “containing”, etc. shall be read expansively and without limitation. The word “comprise” or variations such as “comprises” or “comprising” will accordingly be understood to imply the inclusion of a stated integer or groups of integers but not the exclusion of any other integer or group of integers. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by exemplary embodiments and optional features, modification and variation of the inventions embodied therein herein disclosed may be resorted to by those skilled in the art, and that such modifications and variations are considered to be within the scope of this invention.

The content of all documents and patent documents cited herein is incorporated by reference in their entirety.

1. An organosilica nanoparticle comprising:
a photosensitizer for photodynamic therapy covalently attached thereto, wherein the organosilica nanoparticle is a mesoporous organosilica nanoparticle and the photosensitizer is incorporated within the framework of the nanoparticle.

2. (canceled)

3. The organosilica nanoparticle of claim 1, wherein the organosilica nanoparticle is less than 50 nm in diameter.

4. The organosilica nanoparticle of claim 1, wherein the organosilica nanoparticle is formed by condensation of the photosensitizer with an alkoxysilane.

5. The organosilica nanoparticle of claim 1, wherein the photosensitizer is modified with a silicon-containing group of the formula —Si(OR₆)×(R₇)₃₋ₓ, wherein R₆ and R₇ are independently selected from C₁-C₄ alkyl and C₂-C₄ alkenyl groups, and x is 0, 1, 2, or 3.

6. The organosilica nanoparticle of claim 1, wherein the photosensitizer is a reaction product of phthalocyanine with an alkoxysilane of the formula A—(CH₂)_{y}—Si(OR₆)ₓ(R₇)₃₋ₓ, wherein A is a group reactive with phthalocyanine, x is 0, 1, 2, or 3, and y is 1, 2, or 3.

7. The organosilica nanoparticle of claim 1, wherein the photosensitizer is a phthalocyanine compound of formula (I) or (I′), 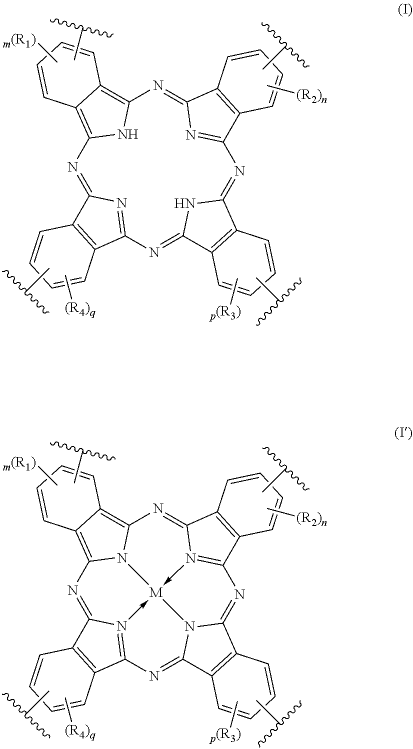
wherein:
M is a metal selected from the group consisting of Zn, Al, Cu, Fe, Mn, Mo, Ni, and V;
R₁, R₂, R₃, and R₄ are each independently C₁-C₆ alkyl;
m, n, p, and q are each independently 0, 1, 2, or 3; and [symbol]
represents a group of formula —NH—B—, wherein B is a silicon-containing linker group that is covalently integrated into the framework of the nanoparticle.

8. The organosilica nanoparticle of claim 1, wherein the organosilica nanoparticle is obtainable using an organosilica precursor of formula (II) or (II′); and an inorganic silica source selected from the group consisting of TMOS, TEOS, and combinations thereof, via silane co-condensation and hydrolysis, 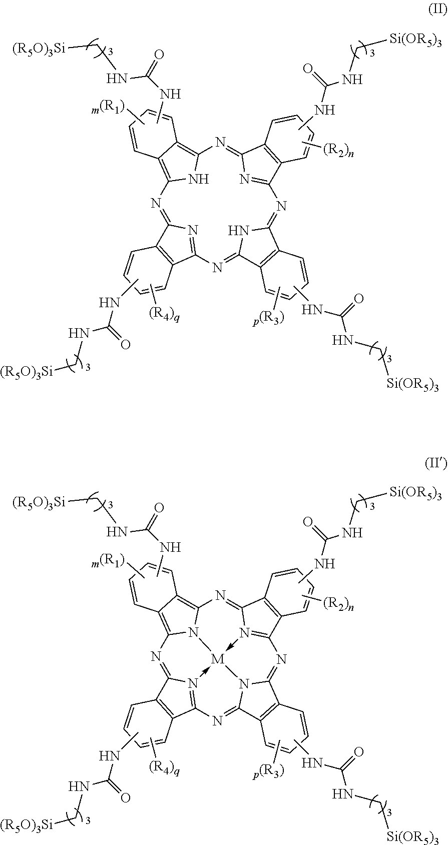
wherein:
M is a metal selected from the group consisting of Zn, Al, Cu, Fe, Mn, Mo, Ni, and V;
R₁, R₂, R₃, R₄, and R₅ are each independently CC₁-C₆ alkyl; and
m, n, p, and q are each independently 0, 1, 2, or 3.

9-12. (canceled)

13. The organosilica nanoparticle of claim 8, wherein the molar ratio of the organosilica precursor of formula (II) or (II′) and the inorganic silica source used for the synthesis of the organosilica nanoparticle is between 1:100 and 1:1000.

14-18. (canceled)

19. A pharmaceutical composition comprising an organosilica nanoparticle and a pharmaceutically acceptable carrier, wherein the organosilica nanoparticle comprises a photosensitizer for photodynamic therapy covalently attached thereto, wherein the organosilica nanoparticle is a mesoporous organosilica nanoparticle and the photosensitizer is incorporated within the framework of the nanoparticle.

20. The pharmaceutical composition of claim 19, wherein the pharmaceutical composition is a topical formulation.

21-22. (canceled)

23. A method for treating a disease, disorder, or condition in a subject, comprising the steps of:
administering to the subject a therapeutically effective amount of an organosilica nanoparticle or a pharmaceutical composition comprising the organosilica nanoparticle and a pharmaceutically acceptable carrier, wherein the organosilica nanoparticle comprises a photosensitizer for photodynamic therapy covalently attached thereto, wherein the organosilica nanoparticle is a mesoporous organosilica nanoparticle and the photosensitizer is incorporated within the framework of the nanoparticle; and
exposing the subject to photoirradiation, thereby treating the disease, disorder, or condition at least partly by photodynamic therapy.

24. The method of claim 23, wherein the photoirradiation is by near-infrared light.

25. The method of claim 23, wherein the disease, disorder, or condition is selected from the group consisting of primary melanoma, metastasized melanoma, basal cell carcinoma, Bowen's disease, actinic keratosis, abnormal scarring (keloid and hypertrophic scars), atoptic dermatitis, warts, pre-malignant non-melanoma skin lesions, and cholangiocarcinoma.

26. The method of claim 23, wherein the disease, disorder, or condition is a skin cancer, and the organosilica nanoparticle or pharmaceutical composition is administered topically.

27. The method of claim 26, wherein the method comprises enhancing skin penetration of the organosilica nanoparticle using a microneedle patch.

28. (canceled)

29. The organosilica nanoparticle of claim 1, further comprising at least one agent encapsulated therein.

30. The organosilica nanoparticle of claim 29, wherein the at least one agent is a compound for the treatment or prevention of a disease, disorder, or condition.

31. The organosilica nanoparticle of claim 30, wherein the disease, disorder, or condition is selected from the group consisting of primary melanoma, metastasized melanoma, basal cell carcinoma, Bowen's disease, actinic keratosis, abnormal scarring (keloid and hypertrophic scars), atoptic dermatitis, warts, pre-malignant non-melanoma skin lesions, and cholangiocarcinoma.

32. The organosilica nanoparticle of claim 29, wherein the at least one agent is selected from the group consisting of antibiotics, steroids, chemotherapeutic drugs, immunomodulators, anti-inflammatory agents, drugs for the treatment of cancer such as BRAF inhibitors, therapeutic peptides or proteins or monoclonal antibodies such as anti-CTLA4 or anti-PD-1 antibodies, siRNAs, and plasmids, or combinations thereof.

33. The organosilica nanoparticle of claim 30, wherein the disease, disorder, or condition is melanoma and the at least one agent is dabrafenib and/or trametinib.
